(19) United States
(12) Patent Application Publication
(10) Pub. No.: US 2026/0123994 A1
(43) Pub. Date: May 7, 2026

(54) SYSTEM AND METHOD FOR ASSESSING INTRACRANIAL ANEURYSMS

(71) Applicant: NEW YORK UNIVERSITY, New York, NY (US)

(72) Inventors: Garigapuram Prithvinath Reddy, New York, NY (US); Srushti Katore, New York, NY (US)

(21) Appl. No.: 18/940,457

(22) Filed: Nov. 7, 2024

(51) Int. Cl. A61B 34/10; A61B 8/06; A61B 8/08; G16H 30/40; G16H 50/30

(52) U.S. Cl. CPC A61B 34/10; A61B 8/06; G16H 30/40; G16H 50/30; A61B 8/488; A61B 2034/105; A61B 2034/108

(57) ABSTRACT

Aspects of the present invention relate to a method for assessing cerebral aneurysms comprising the steps of collecting imaging and blood flow data of an aneurysm and the attached arteries in a subject, creating a 3D model of the aneurysm and the attached arteries from the imaging data, generating blood flow simulations with the 3D model and the blood flow data, extracting one or more parameters from the simulations, processing the one or more parameters to generate one or more stress indices, calculating a risk assessment score for the subject based on a statistical analysis of the one or more parameters and the one or more stress indices.

### CROSS-REFERENCE TO RELATED APPLICATIONS

This application claims priority to U.S. Provisional Application No. 63/597,088, filed on Nov. 8, 2023, incorporated herein by reference in its entirety.

### BACKGROUND OF THE INVENTION

Cerebral aneurysms represent a significant medical concern, affecting a substantial portion of the population. These vascular abnormalities, characterized by a bulging in the arterial wall, pose a serious threat due to their potential to rupture, leading to conditions such as stroke, serious bleeding, increased intracranial pressure, and even death. Their prevalence, particularly in the anterior circulation near arterial bifurcations, necessitates reliable methods for diagnosis, assessment, and treatment planning.

Traditionally, the diagnosis and assessment of cerebral aneurysms have relied on medical imaging techniques such as CT scans, CTA, MRI, and MRA. While these methods are valuable for detecting aneurysms, they often provide limited insights into the dynamic behavior of blood flow within aneurysms and their associated arteries. Moreover, most aneurysms are discovered only after they rupture, leading to emergency situations with limited time for comprehensive evaluation and treatment decisions.

Computational Fluid Dynamics (CFD) is a well-established technique used in various fields to simulate and predict the behavior of fluids and structures. In recent years, the application of CFD in the medical field has shown promise in providing a deeper understanding of complex phenomena such as cerebral aneurysms. Hemodynamic parameters, which describe blood flow dynamics, have emerged as critical metrics for assessing aneurysms. These parameters, including Flow Velocity, Pressure Distribution, Wall Shear Stress (WSS), Time Average Wall Shear Stress (TAWSS), Oscillatory Shear Index (OSI), and Relative Residence Time (RRT), offer valuable insights into aneurysm progression, growth, rupture risk, and treatment outcomes.

While CFD analysis has the potential to revolutionize the assessment of cerebral aneurysms, its widespread adoption in clinical practice has been hindered by several challenges. CFD is computationally intensive, requiring complex simulations tailored to individual patient data. Furthermore, the extraction of subject-specific 3D models from medical images, real-time flow and pressure data from Doppler Ultrasonography, and the subsequent integration of these components into a cohesive analysis pipeline require manual, time-consuming, and error-prone processes.

The existing methods for assessing cerebral aneurysms often lack the integration of real-time patient-specific data, hindering their accuracy and clinical utility. Additionally, there is a need for efficient automation of the entire CFD analysis process, reducing reliance on human intervention and enabling real-time risk assessment and treatment decision-making.

Thus, there is a need in the art for an improved system and methodology that automates and streamlines the assessment of cerebral aneurysms, providing clinicians with accurate, patient-specific data for risk assessment and treatment planning. The present invention meets this need.

### SUMMARY OF THE INVENTION

Aspects of the present invention relate to a method for assessing cerebral aneurysms comprising the steps of collecting imaging and blood flow data of an aneurysm and the attached arteries in a subject, creating a 3D model of the aneurysm and the attached arteries from the imaging data, generating blood flow simulations with the 3D model and the blood flow data, extracting one or more parameters from the simulations, processing the one or more parameters to generate one or more stress indices, calculating a risk assessment score for the subject based on a statistical analysis of the one or more parameters and the one or more stress indices.

In some embodiments, the imaging data may be collected from any of CT Cerebral Angiogram, MRI, MRA, 3D DSA or 4D DSA. In some embodiments, the blood flow data comprises real-time flow velocity and pressure data from Doppler Ultrasonography.

In some embodiments, the one or more parameters comprise a hemodynamic parameter and/or a morphological parameter. In some embodiments, the hemodynamic parameter is selected from the group consisting of: Pressure, Pressure Distribution, Flow Velocity, Velocity profile, Wall Shear Stress (WSS), mean maximum WSS (MWSS), mean parent vessel WSS (PTWSS), mean normalized WSS (NWSS), WSS gradient (WSSG), Transverse WSS (TWSS), Aneurysm formation indicator (AFI), Oscillation velocity index (OVI), Gradient oscillatory number (GON), Relative residence time (RRT), or mean oscillatory shear index (OSI). In some embodiments, the morphological parameter is selected from the group consisting of: aneurysm size, aspect ratio (AR), size ratio (SR), ellipticity index (EI), undulation index (UI), nonsphericity index (NSI), shape of aneurysm, bottleneck factor (BNF), vessel angle, parent artery diameter, aneurysm neck width, aneurysm height, or aneurysm width. In some embodiments, the stress indices comprise any of Time Average Wall Shear Stress (TAWSS), Oscillatory Shear Index (OSI), and Relative Residence Time (RRT).

In some embodiments, the step of calculating the risk score comprises a statistical analysis to correlate at least one of the one or more parameters and stress indices with one or more medical factors of the subject. In some embodiments, the one or more medical factors comprise any of hypertension, sex, smoking, age, medical history, aneurysm type, aneurysm location, rupture status, multiple aneurysms, genetic predisposition affecting the aneurysm condition, injury or trauma to blood vessels, complications from some types of blood infections, blood lipid levels, glucose levels, and indication for diabetes.

In some embodiments, the method further comprises the step of treating the subject with one or more treatments based on the outcome of the risk assessment score. In some embodiments, the treatment comprises implantation of a stent. In some embodiments, the method further comprises the step of assessing the implementation and effectiveness of stent by repeating any previous steps to calculate a treatment assessment score.

In some embodiments, the step of creating the 3D model further comprises applying boundary conditions to the imaging data to create a volume, applying a fluid governing equation to the volume, and providing parameters from the blood flow data to the fluid governing equation. In some embodiments, the step of generating the blood flow simulation comprises providing one or more data inputs from flow velocity and pressure values of the blood flow data. In some embodiments, the statistical analysis is performed in a neural network trained with imaging and blood flow data associated with vessel occlusion.

Aspects of the present invention relate to a system for assessing cerebral aneurysms in a subject comprising a non-transitory computer-readable medium with instructions stored thereon, that when executed by a processor perform the steps of collecting imaging and blood flow data of an aneurysm and the attached arteries in a subject, creating a 3D model of the aneurysm and the attached arteries from the imaging data, generating blood flow simulations with the 3D model and the blood flow data, extracting one or more parameters from the simulations, processing the one or more parameters to generate one or more stress indices, calculating a risk assessment score for the subject based on a statistical analysis of the one or more parameters and the one or more stress indices.

In some embodiments, the imaging data may be collected from any of CT Cerebral Angiogram, MRI, MRA, 3D DSA, or 4D DSA. In some embodiments, the blood flow data comprises real-time flow velocity and pressure data from Doppler Ultrasonography. In some embodiments, the one or more parameters comprise a hemodynamic parameter and/or a morphological parameter. In some embodiments, the hemodynamic parameter is selected from the group consisting of: Pressure, Pressure Distribution, Flow Velocity, Velocity profile, Wall Shear Stress (WSS), mean maximum WSS (MWSS), mean parent vessel WSS (PTWSS), mean normalized WSS (NWSS), WSS gradient (WSSG), Transverse WSS (TWSS), Aneurysm formation indicator (AFI), Oscillation velocity index (OVI), Gradient oscillatory number (GON), Relative residence time (RRT), or mean oscillatory shear index (OSI).

In some embodiments, the morphological parameter is selected from the group consisting of: aneurysm size, aspect ratio (AR), size ratio (SR), ellipticity index (EI), undulation index (UI), nonsphericity index (NSI), shape of aneurysm, bottleneck factor (BNF), vessel angle, parent artery diameter, aneurysm neck width, aneurysm height, or aneurysm width. In some embodiments, the stress indices comprise any of Time Average Wall Shear Stress (TAWSS), Oscillatory Shear Index (OSI), and Relative Residence Time (RRT). In some embodiments, the statistical analysis is performed in a neural network trained with imaging and blood flow data associated with vessel occlusion.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A depicts an illustrative computer architecture for a computer for practicing the various embodiments of the invention.

FIG. 1B depicts an exemplary method for assessing cerebral aneurysms according to aspects of the present invention.

FIG. 2 is a flowchart depicting an exemplary method for assessing cerebral aneurysms according to aspects of the present invention.

FIG. 3 is a flowchart depicting exemplary processes involved in an exemplary method comprising Computational Fluid Dynamics (CFD).

FIG. 4A depicts a 3D model of a blood vessel wherein the geometry is shown as a faceted body. FIG. 4B depicts a 3D model of a blood vessel wherein the geometry is shown as an extracted volume.

FIG. 5A depicts the implementation of a body sizing mesh around a 3D model representing any of a region of interest (ROI), site of interest, a blood vessel, a cerebral aneurysm, and/or an intracranial aneurysm (IA) according to aspects of the present invention. FIG. 5B depicts the implementation of an inflation mesh within a 3D model representing any of a ROI, site of interest, a blood vessel, a cerebral aneurysm, and/or an intracranial aneurysm (IA) according to aspects of the present invention.

FIG. 6 is a plot showing the results for Wall Shear Stress Distribution across a 3D model according to aspects of the present invention.

FIGS. 7A & 7B are a flowchart of an exemplary software integration for a method for assessing cerebral aneurysms according to aspects of the present invention.

FIG. 8 is a flowchart of an exemplary method for assessing cerebral aneurysms comprising Artificial Intelligence (AI)/Machine Learning (ML) according to aspects of the present invention.

FIG. 9 is an image depicting medical imaging of a cerebral aneurysm.

FIG. 10A is an image depicting the important locations in the brain where aneurysms can occur. FIG. 10B is an image depicting various types of cerebral aneurysms.

FIG. 11 is a flowchart depicting an exemplary method for assessing aneurysms according to aspects of the present invention.

FIG. 12 is a plot showing an inlet velocity profile in two periods.

FIG. 13 is a plot showing a velocity profile for a 3D model of an ROI according to aspects of the present invention.

FIG. 14 is a plot showing a pressure distribution for a 3D model of an ROI according to aspects of the present invention.

FIG. 15 is a plot showing a wall shear stress distribution for a 3D model of an ROI according to aspects of the present invention.

FIG. 16 is a flowchart depicting a diagnosis method for treatment prescription.

FIG. 17 is a flowchart depicting an exemplary method for diagnosis of aneurysms in real-time according to aspects of the present invention.

FIG. 18 depicts a solid 3D model for the site of an intracranial aneurysm (IA).

FIG. 19 shows the implementation of a body sizing mesh around a solid model of an IA according to aspects of the present invention.

FIG. 20 shows a plot for Wall Shear Stress (WSS) distribution across the walls of the ruptured IA according to aspects of the present invention.

FIG. 21 is a flowchart depicting an exemplary training model comprising a stack classifier according to aspects of the present invention.

### DETAILED DESCRIPTION

It is to be understood that the figures and descriptions of the present invention have been simplified to illustrate elements that are relevant for a clear understanding of the present invention, while eliminating, for the purpose of clarity many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, or +0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Computing Device

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled, or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, virtual machines, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

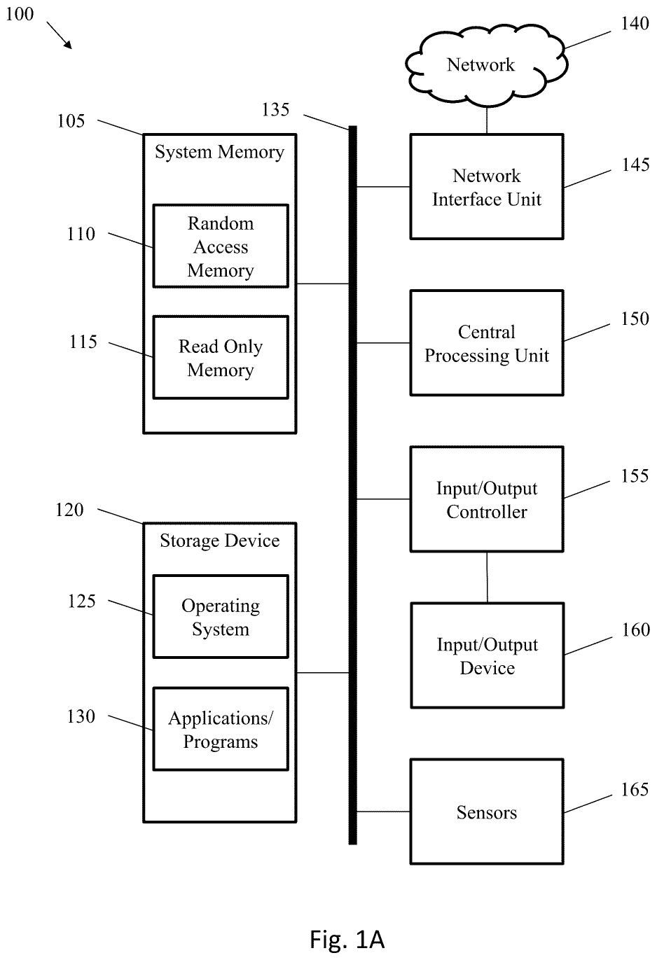

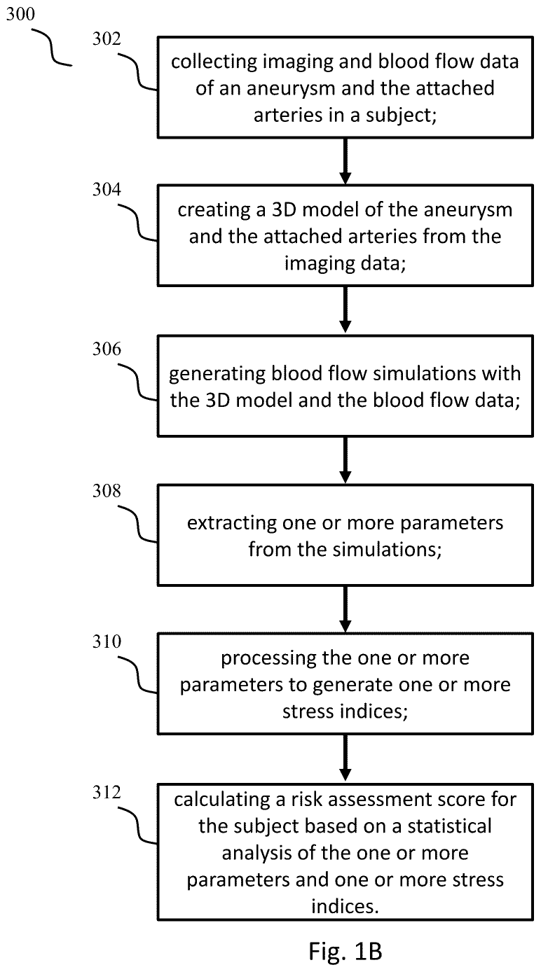

FIG. 1A and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 1A depicts an illustrative computer architecture for a computer 100 for practicing the various embodiments of the invention. The computer architecture shown in FIG. 1A illustrates a conventional personal computer, including a central processing unit 150 (“CPU”), a system memory 105, including a random access memory 110 (“RAM”) and a read-only memory (“ROM”) 115, and a system bus 135 that couples the system memory 105 to the CPU 150. A basic input/output system containing the basic routines that help to transfer information between elements within the computer, such as during startup, is stored in the ROM 115. The computer 100 further includes a storage device 120 for storing an operating system 125, application/program 130, and data. The computer architecture may in certain embodiments physically reside in medical equipment found in hospitals and surgical suites, or otherwise communicatively coupled with the medical equipment.

The storage device 120 is connected to the CPU 150 through a storage controller (not shown) connected to the bus 135. The storage device 120 and its associated computer-readable media provide non-volatile storage for the computer 100. Although the description of computer-readable media contained herein refers to a storage device, such as a hard disk or CD-ROM drive, it should be appreciated by those skilled in the art that computer-readable media can be any available media that can be accessed by the computer 100.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 100 may operate in a networked environment using logical connections to remote computers through a network 140, such as TCP/IP network such as the Internet or an intranet. The computer 100 may connect to the network 140 through a network interface unit 145 connected to the bus 135. It should be appreciated that the network interface unit 145 may also be utilized to connect to other types of networks and remote computer systems.

The computer 100 may also include an input/output controller 155 for receiving and processing input from a number of input/output devices 160, including medical equipment found in hospitals and surgical suites such as cameras and imaging equipment, or more generally including a keyboard, a mouse, a touchscreen, a camera, a microphone, a controller, a joystick, or other type of input device. Similarly, the input/output controller 155 may provide output to a display screen, a printer, a speaker, or other type of output device. The computer 100 can connect to the input/output device 160 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 120 and/or RAM 110 of the computer 100, including an operating system 125 suitable for controlling the operation of a networked computer. The storage device 120 and RAM 110 may also store one or more applications/programs 130. In particular, the storage device 120 and RAM 110 may store an application/program 130 for providing a variety of functionalities to a user. For instance, the application/program 130 may comprise many types of programs such as a word processing application, a spreadsheet application, a desktop publishing application, a database application, a gaming application, internet browsing application, electronic mail application, messaging application, and the like. According to an embodiment of the present invention, the application/program 130 comprises a multiple functionality software application for providing word processing functionality, slide presentation functionality, spreadsheet functionality, database functionality and the like.

The computer 100 in some embodiments can include a variety of sensors 165 for monitoring the environment surrounding and the environment internal to the computer 100. These sensors 165 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### System and Method for Assessing Intracranial Aneurysms

The system and method disclosed herein provide an AI-enhanced approach for assessing intracranial aneurysms by combining 3D modeling, Computational Fluid Dynamics (CFD) simulations, and machine learning. The system uses patient-specific imaging data, including Doppler Ultrasonography, CT, and MRI, to generate a 3D model of the aneurysm and surrounding vasculature. CFD simulations are applied to assess hemodynamic parameters such as Wall Shear Stress (WSS) and Oscillatory Shear Index (OSI), which are indicators of aneurysm rupture risk. These parameters are then used to train an AI model that analyzes patient-specific risk factors, outputs one or more risk assessments (e.g., a stress index or Risk of Rupture (RoR)), and aids clinicians in making informed treatment decisions. The system's continuous feedback mechanism enables model updates as new clinical data is acquired, improving accuracy over time.

Aspects of the present invention relate to a system and method for assessing cerebral aneurysms using Computational Fluid Dynamics (CFD) analysis. In some embodiments, the disclosed system and method performs the steps of obtaining, capturing, or measuring data on one or more parameters of intracranial aneurysms (e.g., hemodynamic parameters, morphological parameters) from a patient, and combining the parameters with patient data (e.g., medical factors), thereby providing an accurate risk assessment for the patient enabling early treatment decision-making, and insights into the nature and behavior of these medical conditions. In some embodiments, the risk assessment comprises a statistical analysis of hemodynamic parameters, morphological parameters, and patient medical data (e.g. medical factors) related to an aneurysm or vasculature in a subject.

A system for assessing intracranial aneurysms is disclosed herein. In some embodiments, the system connects to a computing device (e.g., computer 100) and/or operates as a software residing on a computing device. In some embodiments, the disclosed system comprises a primary software or platform that utilizes or connects with one or more commercial-off-the-shelf (COTS) software (e.g., DICOM viewer, image processing software, ANSYS™, OsiriX™, MATLAB, other licensed software, and any combinations thereof). In some embodiments, the system comprises one or more displays and/or provides a user interface (UI) or graphical user interface (GUI) to a user. In some embodiments, the UI displays the vasculature of a subject and/or any of the disclosed parameters, measured values, calculated values or scores. In some embodiments, the GUI displays patient or subject data such as medical history data. In some embodiments, the GUI provides at least one visual representation or image of a target site, area of interest, or region of interest (ROI) in a subject. In some embodiments, the GUI provides a representation of an aneurysm, and/or one or more predicted aneurysms. In some embodiments, the representation or image comprises any of medical imaging data, a 3D reconstruction of medical imaging data, a 3D model of an ROI, meshes applied to the 3D reconstruction or 3D model, volumes applied to the 3D reconstruction or 3D model, flow simulations, aneurysm predictions, stress indices and/or rupture risk score. In some embodiments, the software provides alerts based on thresholds of the measured parameters, or simulated and calculated values or scores. In some embodiments, the software provides interactive visuals of the simulations to aid with further interpretation.

In some embodiments, the system comprises a non-transitory computer-readable medium with instructions stored thereon, that when executed by a processor perform any disclosed methods and/or steps.

Aspects of the present invention relate to a method for assessing intracranial aneurysms. Referring now to FIG. 1B, shown is exemplary method 300 for assessing intracranial aneurysms, comprising the steps of 302 collecting imaging and blood flow data of an aneurysm and the attached arteries in a subject; 304 creating a 3D model of the aneurysm and the attached arteries from the imaging data; 306 generating blood flow simulations with the 3D model and the blood flow data; 308 extracting one or more parameters from the simulations; 310 processing the one or more parameters to generate one or more stress indices; 312 calculating a risk assessment score for the subject based on a statistical analysis of the one or more stress indices.

In some embodiments, the imaging data may be collected from a CT Cerebral Angiogram, an MRI, an MRA, a 3D DSA, or a 4D DSA. In some embodiments, the blood flow data comprises real-time flow velocity and pressure data from a Doppler Ultrasonography. In some embodiments, the one or more parameters comprise any of hemodynamic parameters and/or a morphological parameters. In some embodiments, the one or more hemodynamic parameters comprises any of: Pressure, Pressure Distribution, blood Flow Velocity, blood flow Velocity profile, Wall Shear Stress (WSS), mean maximum WSS (MWSS), mean parent vessel WSS (PTWSS), mean normalized WSS (NWSS), WSS gradient (WSSG), Transverse WSS (TWSS), Aneurysm formation indicator (AFI), Oscillation velocity index (OVI), Gradient oscillatory number (GON), Relative residence time (RRT), mean oscillatory shear index (OSI), or Endothelial cell activation potential (ECAP).

In some embodiments, the morphological parameter is selected from any of aneurysm size, aspect ratio (AR), size ratio (SR), ellipticity index (EI), undulation index (UI), nonsphericity index (NSI), shape of aneurysm, bottleneck factor (BNF), vessel angle, parent artery diameter, aneurysm neck width, aneurysm height, or aneurysm width. In some embodiments, the stress indices comprise any of Time Average Wall Shear Stress (TAWSS), Oscillatory Shear Index (OSI), Relative Residence Time (RRT) or Endothelial cell activation potential (ECAP).

In some embodiments, the step of calculating the risk score comprises a statistical analysis to correlate the hemodynamic parameters and stress indices with one or more factors in the subject (e.g., medical factors). In some embodiments, method 300 comprises the step of providing one or more medical factors of the subject. In some embodiments, the step of calculating a risk assessment score for the subject comprises a statistical analysis of the one or more parameters, the one or more stress indices and/or the one or more medical factors of the subject. In some embodiments, the one or more factors (e.g., medical factors) comprise any of hypertension, sex, smoking, age, medical history, aneurysm type, aneurysm location, rupture status, multiple aneurysms, genetic predisposition affecting the aneurysm condition, injury or trauma to blood vessels, blood lipid levels, diabetes (glucose levels) and complications from some types of blood infections.

In some embodiments, method 300 further comprises the step of treating the subject with one or more treatments based on the outcome of the risk assessment score. In some embodiments, the treatment comprises monitoring the subject over a period. In some embodiments, the treatment comprises implantation of a stent or any intravascular procedure or intervention know by one of ordinary level of skill in the art. In some embodiments, method 300 further comprises the step of assessing the implementation and effectiveness of the stent by repeating any previous steps in order to calculate a final treatment assessment or outcome score.

In some embodiments, the step of creating the 3D model further comprises applying boundary conditions to the imaging data to create a volume, applying a fluid governing equation (e.g., the Navier Stokes Equation) to the volume, and providing parameters from the blood flow data to the fluid governing equation. In some embodiments, the step of generating the blood flow simulation comprises providing one or more data inputs from flow velocity and pressure values of the blood flow data. In some embodiments, the risk assessment will be provided based on the statistical analysis of hemodynamic and morphological parameters and patient medical data. The disclosed system and method may be used to calculate any number of assessments or logistical calculations. In some embodiments, a risk assessment is performed by calculating one or more calibrated risk probabilities. Any known vessel occlusion and/or aneurysm scoring or risk assessment may be calculated using the disclosed system, and may include any known methods, calculations, and related equations for aneurism identification and/or assessment such as Risk of Rupture (RoR) as would be known by one of ordinary level of skill in the art. In some embodiments, one or more risk scores, risk probabilities, and/or calibrated risk probabilities may be calculated using key risk factor coefficients to calculate one or more calibrated risk probabilities with an odds equation (Equation 1). In some embodiments, the calculation of the probabilities includes incorporating multimodal patient specific data framework comprising morphology parameters, hemodynamic parameters, and medical factors, and calculates a risk score scaled between 1 and 10. In some embodiments, a risk assessment score is calculated with an odds equation,

$$Odds = {\sum }^{″} Coeff * ( Variable ) + c \qquad Equation 1$$

where, Variable={morphology features, Hemodynamic features, medical factors}, c is a constant or intercept based on the odds or coefficient of the listed variables, and uses key risk factors' coefficients in order to calculate one or more calibrated risk probabilities or risk scores.

Aspects of the invention relate to a machine learning algorithm, machine learning engine, machine learning model, or neural network. In various embodiments, the machine engine may include supervised, semi-supervised and unsupervised learning. In the case of supervised learning, algorithms may include nearest neighbor, naïve Bayes, decision trees, support vector machines (SVM), and neural networks. In various embodiments, models may be trained by updating parameters based on various attributes of a blood vessel and/or vessel occlusion, for example hemodynamic parameter data, morphological parameter data, imaging and blood flow data, blood vessel parameters, occlusion factors, and subject factors, and may output an identified anatomical structure or occlusion, aneurysm growth, progression of the aneurysm, and/or a risk assessment (an RoR) or report based on the attributes. In various embodiments, the supervised or semi-supervised model may be updated with hyperparameters for a deep learning model. In a preferred embodiment, a neural network may be trained by updating parameters based on various attributes of a blood vessel, for example vessel wall thickness, vessel wall elasticity and flow dynamics in the vessel walls and may output a risk assessment report based on any of the attributes.

In some embodiments, attributes may include hemodynamic parameter data, stress indices, vessel wall thickness, vessel wall elasticity and flow dynamics in the vessel walls, hypertension, sex, smoking, age, medical history, aneurysm type, aneurysm location, rupture status, multiple aneurysms, morphological parameters like SizeRatio of aneurysm, genetic predisposition affecting the aneurysm condition, injury or trauma to blood vessels, and complications from some types of blood infections and the growth, rupture risk, and progression of the aneurysm. The resulting anatomical structure (e.g., blood vessel) and/or vessel occlusion may then be judged according to one or more binary classifiers or quality metrics, and the weights of the attributes may be optimized to maximize the average binary classifiers or quality metrics. In this manner, a neural network can be trained to predict and optimize for any binary classifier or quality metric that can be experimentally measured. Examples of binary classifiers or quality metrics that a neural network can be trained on include blood flow data, hemodynamic parameter data, and any other suitable type of quality metric that can be measured, using any type of machine learning algorithm (e.g., SVM), calibrated classifier, random forest and/or regression. In some embodiments, the neural network may have multi-task functionality and allow for simultaneous prediction and optimization of multiple quality metrics.

In embodiments that implement supervised or semi-supervised models, such as a neural network, a query may be performed in various ways. A query may request the neural network identify a blood vessel and/or vessel occlusion to increase a desirable parameter, for example blood flow profile, velocity or pressure, WSS, or any other relevant parameter known by one of ordinary level of skill in the art correlated with biological function. A supervised or semi-supervised learning model of the present invention may identify one or more anatomical structures (e.g., blood vessels) and/or injuries or conditions (e.g., vessel occlusions) whose predicted blood flow velocity or pressure (as evaluated by the neural network) decreases or increases, respectively, thereby indicating the condition of the vessel and/or presence or severity of an occlusion. As contemplated herein, a predicted occlusion may be any stroke (Cardiovascular, Intracranial and/or Cerebral), subcranial hemorrhage, aneurysms in different areas such as abdominal, plaque deposition in arteries, as well as the study of blood vessels and arteries with the same hemodynamic parameters.

In some embodiments, the supervised or semi-supervised learning model may be updated by training the model using a value of the desirable parameters associated with hemodynamic parameters (e.g., input blood flow velocity and/or pressure), and/or treatments for vessel occlusion. In the case of the neural network, this may be referred to as the input layer. Updating the model in this manner may improve the ability of the model in identifying an anatomical structure or occlusion, proposing a treatment or optimal hemodynamic parameter (e.g., blood flow velocity and/or pressure). In the case of the neural network, this may be referred to as the output layer. In some embodiments, training the model may include using a value of the desirable parameter associated with known anatomical structures, treatments or hemodynamic parameters (e.g., blood flow velocity and/or pressure measurements associated with biological function), or known parameters or values that indicate vessel occlusion. For example, in some embodiments, training the model may include predicting a value of the desirable parameter for the inputted blood flow velocity, comparing the predicted value to the corresponding value associated with a known blood flow velocity, and training the model based on a result of the comparison. If the predicted value is the same or substantially similar to the observed value, then the neural network may be minimally updated or not updated at all. If the predicted value differs from that of the known blood flow velocity, then the model may be substantially updated to better correct for this discrepancy. Regardless of how the model is retrained, the retrained model may be used to propose additional risk assessment scores, treatments, or blood flow velocities and/or pressures, or identify injuries, conditions and/or anatomical structures.

Although the techniques of the present application may be presented in the context of the assessment of vasculature and vascular occlusion, and/or occlusion prediction and treatment thereof, it should be appreciated that this is a non-limiting application of these techniques as they can be applied to any types of parameters or attributes associated identification of anatomical structures with vasculature and vessel occlusion, for example hemodynamic parameter data, stress indices, vessel wall thickness, vessel wall elasticity and flow dynamics in the vessel walls, flow simulations, imaging data, one or more medical factors, hypertension, sex, smoking, age, medical history, aneurysm type, aneurysm location, rupture status, multiple aneurysms, genetic predisposition affecting the aneurysm condition, injury or trauma to blood vessels, and complications from some types of blood infections and the growth, rupture risk (e.g., RoR), and progression of the aneurysm. Depending on the type of data used to train the model, the neural network can be optimized for different types of reports. In this manner, a neural network can be trained to identify anatomical structures, injury types and risk factors associated with vessel occlusion and assess the data generated from the methods to provide insight into the underlying biological conditions of the vessels and walls, as well provide a risk assessment such as an RoR or one or more stress indices. Querying the model may include inputting an a 3D model of a region of interest in a subject, imaging data, blood flow data and/or flow simulations thereof. The model may have been previously trained using spatial and morphological characteristics associated with different conditions or injuries related to vessel occlusions (e.g., aneurysms) and flow simulations and blood flow data associated with the conditions or injuries. The query to the model may be for a predicted aneurysm type or location, or may include one or more medical factors for the subject or blood flow data. An identified anatomical structure, flow simulation, predicted flow, predicted rupture, risk assessment score and/or a treatment recommendation or outcome score may be received from the model in response to the query.

The techniques described herein are associated with iteratively querying a model by inputting a 3D model of a vessel, 3D model of a vessel occlusion, flow simulations or fluid dynamics in the vessel or occlusion, or subject or medical factors, receiving an output from the model that has a significance and correlation report of the inputs, and successively providing the report as an input to the model, can be applied to other machine learning applications. Such techniques may be particularly useful in applications where a final output having treatment validation assessment is desired. Such techniques can be generalized for identifying a series of discrete attributes by applying a model trained using data relating the discrete attributes to a characteristic of a series of the discrete attributes. In the context of the assessment of vasculature and vascular occlusion, and the prediction of an occlusion, the discrete attributes may include flow simulation data, hemodynamic parameters, blood flow data (e.g., doppler ultrasonography inputs), hypertension, smoking, age, medical history, and genetic predisposition that affect the condition for aneurysm, or any other disclosed parameters or attributes.

In some embodiments, the disclosed method may utilize machine learning methods such as Hyper Parameter Tuning to engineer the best combination of factors and create an accurate model that aligns with the actual data. In some embodiments, to deal with the dataset with fewer rows, dimensionality reduction techniques may be applied like Principal Component Analysis (PCA) and Linear Discriminant Analysis (LDA) to transform data into lower-dimensional space while preserving variance. If the classification methods are ineffective in raising the bar, regularization techniques may be utilized to maintain the context of the data while shrinking down less relevant data points. These techniques do not change classification results significantly, but speed up computation without adding too many features, and avoid overfitting.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Computational Analysis for Effects on Hemodynamic Parameters Based on the Location of Cerebral Aneurysms

An aneurysm is a bulging on the artery's wall produced by the weakening of the muscles within the vascular wall. Since arteries transport blood under high pressure, they are prone to aneurysm development. Cerebral aneurysms can affect about 1.5 to 5 percent of the general population. They are more prevalent in women than men and most frequently affect persons between 30 and 60. Because of the ongoing high-pressure loading and unloading, the arteries within the brain are more prone to aneurysm development. A ruptured aneurysm in the brain can result in a stroke, serious bleeding, increased pressure on the brain, and in the worst-case scenario, death. The majority of brain aneurysms are saccular and range in size from 0.5 mm to more than 25 mm. They are most commonly seen in the anterior circulation near the bifurcation of an artery. They are diagnosed by utilizing medical imaging procedures such as CT scans, CTA, MRI, and MRA, among others. Although most cerebral aneurysms are discovered after rupturing, causing medical emergencies, they are also found when patients undergo scans for other conditions.

Computational Fluid Dynamics (CFD) is applied in a multitude of fields. It provides accurate simulations and predictions for different types of fluids and structures. Its use in the medical field is relatively new. Cerebral Aneurysms are a complex phenomenon, thus applying CFD to cerebral aneurysms provides new insights. The metrics against which cerebral aneurysms are assessed are well known in the art. These metrics are termed Hemodynamic parameters. They give insights into the blood flow parameters and the effects are observed over some cardiac cycles. These insights can be further analyzed against the different factors that have an effect on the progression, growth, rupture, and treatment of an aneurysm.

Gaining insights into such flow dynamics provides essential information that helps in assessing the nature of the aneurysm, predicting its behavior, and helping in making informed treatment decisions eliminating uncertainty. It is essential that this data is supported with the real-time measurements (e.g., flow and pressure metrics) for it to be accurate and custom to each subject.

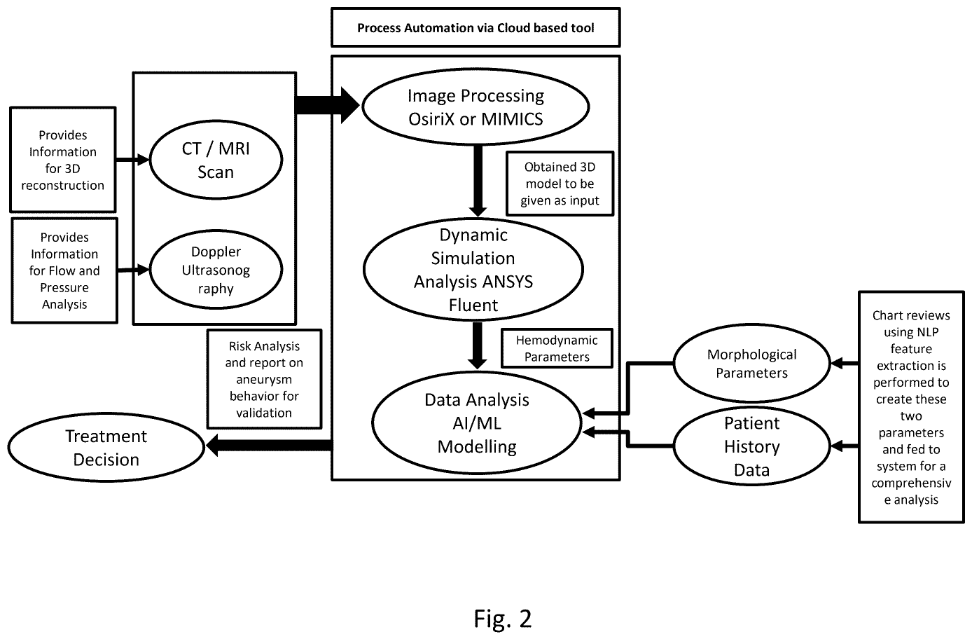

The system and method disclosed in this example proposes CFD analysis for cerebral aneurysms in a clinical setting as a tool for treatment decision-making. As CFD is computationally heavy and case-specific, an automated system and method is disclosed herein that can convert subject-specific data by extracting a 3D model, and generate a solution using real-time flow metrics of a subject. Further, the solution generated by the system and method is used for statistical analysis to provide risk assessment for each subject. Automating the system and method provides ease of use in a clinical setting along with comprehensive data that is currently unavailable for assessment. FIG. 2 is a flowchart showing an overview of an exemplary method according to aspects of the present invention. The system and method may operate on and at least reside in part on any suitable computing device or computer. An exemplary method is further discussed herein.

CT Cerebral Angiogram/MRI/3D DSA: In some embodiments, the disclosed method comprises the step of the patient data being collected using imaging techniques such as CT, MRI, MRA, 3D DSA, and/or 4D DSA. In some embodiments, imaging techniques such as image segmentation are applied to the data to extract a 3D model of the aneurysm and its attached arteries.

Doppler Ultrasonography: In some embodiments, the disclosed method comprises providing real-time measurements or metrics (e.g., flow velocity and pressure inputs), and generating an accurate representation of the model specific to each subject. In some embodiments, doppler ultrasonography generates a velocity profile of the blood and its pressure at the target arteries, which is then fed to the system as inputs.

Process Automation: The disclosed system and method involves three major components, image segmentation, solution generation, and statistical analysis.

Image Processing: Any suitable image processing software may be used to process the images. In this example, the software for conversion of Digital Imaging and Communications in Medicine (DICOM) images obtained through imaging to 3D models to be used for simulation was Osirix™, which has the capabilities and necessary image segmentation techniques for tracing the arteries' geometry and generating 3D models. Using an Application Programming Interface (API), the software minimizes the human intervention necessary for the required techniques. The software was able to identify the Region of Interest (ROI) based on the inputs given to the system. The generated 3D model may be outputted or saved in any 3D file format (e.g., an .stl file format) which may be further used the system and method (e.g., for generating a simulation).

Simulation and Analysis: The disclosed system and method may use any suitable fluid dynamics software. In this example, the software used was the ANSYS™ software platform, which may be integrated into the disclosed system and method for generating accurate simulations (e.g., fluid or flow simulations). The simulations and analysis thereof are used to generate solutions for a subject. The solution generation process comprises of three major components which are further discussed herein. The solution obtained is in numerical format thus making it ideal for further statistical analysis. The fluid dynamics software is also used by the disclosed system and method to simulate blood flow through the arteries and provides an accurate representation of the physical contours of the ROI. In some embodiments, the disclosed system and method calculates or predicts how the aneurysm will be affected given a specific treatment, or subject, or provides a decision assistance tool.

Data Analysis: In some embodiments, the data collected from the fluid simulation software (e.g., ANSYS™) is processed to generate hemodynamic parameters (e.g., Indices of Wall Shear Stress). The parameters provide an accurate graphical representation for risk assessment. In some embodiments, the data is further tested for statistical significance using any suitable programming platform (e.g. Python). In some embodiments, the programming platform is used for generating an assessment report to aid in proper treatment decision-making.

Boundary conditions: Blood is a Non-Newtonian fluid, demonstrating a pulsatile flow mimicking the pumping of the heart. To simplify the complexities involved in the modeling of a Non-Newtonian fluid, the fundamental governing equations used are continuity and the Navier-Stokes which are further simplified to incorporate into the Carreau model. For the boundary conditions, each model may comprise custom inputs for the velocity and pressure parameters obtained from the Doppler Ultrasonography procedure. In some embodiments, the wall conditions are customized for each model.

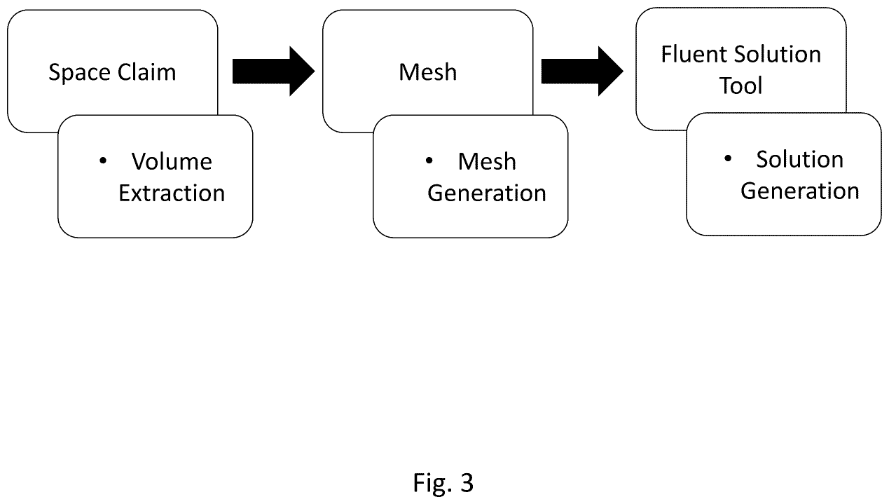

Process involved: In some embodiments, the CFD procedure is carried out in three stages using the fluid dynamics software (e.g., ANSYS™ platform). FIG. 3 shows a flowchart for exemplary processes involved in the CFD procedure. In some embodiments, these processes are automated using APIs to minimize human intervention.

The processes are discussed herein.

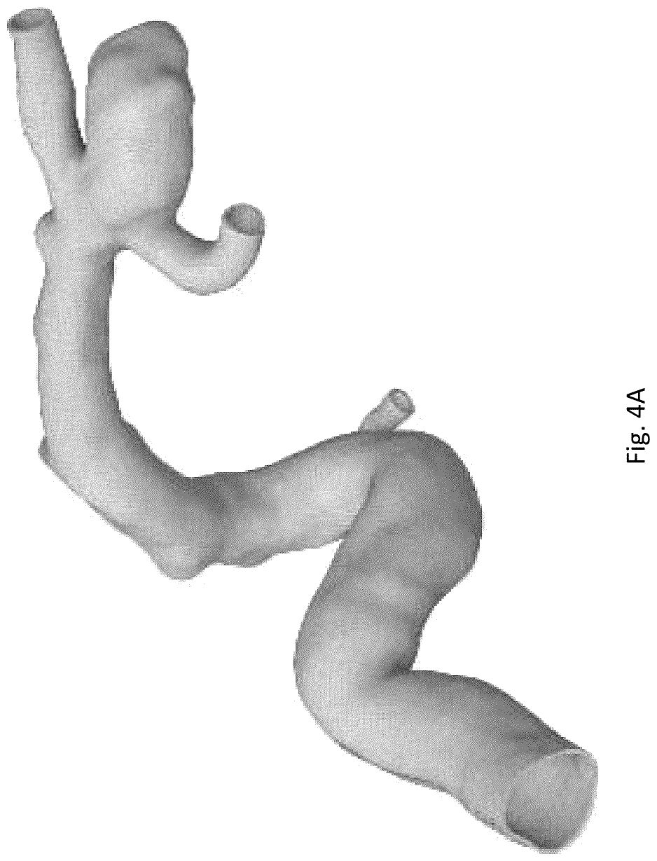

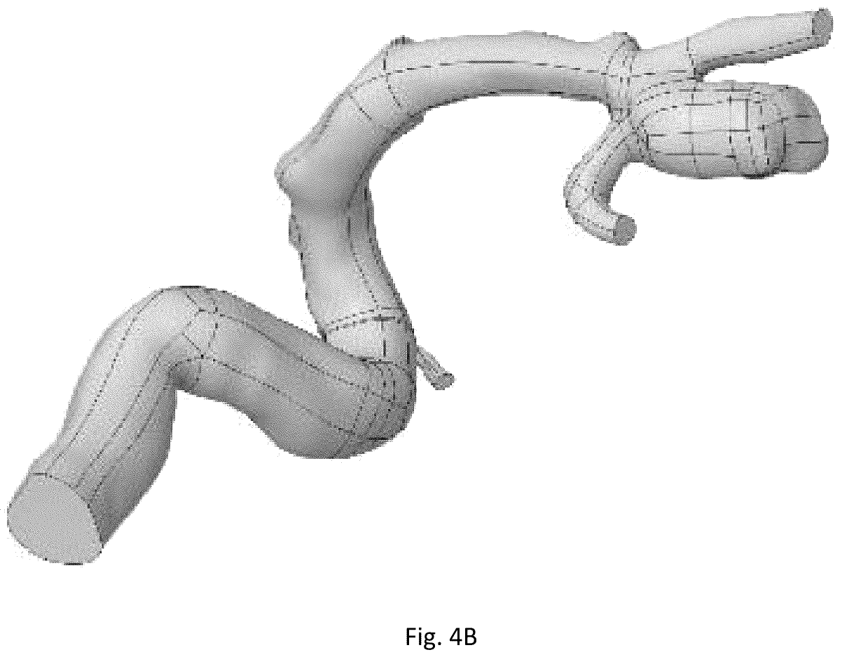

Space Claim: In some embodiments, the volume extraction process is carried out in SpaceClaim, ANSYS™. It is automated using Python scripts to perform the extraction for each model. FIG. 4A depicts a 3D model of a blood vessel wherein the geometry is shown as a faceted body. FIG. 4B depicts a 3D model of a blood vessel wherein the geometry is shown as an extracted volume.

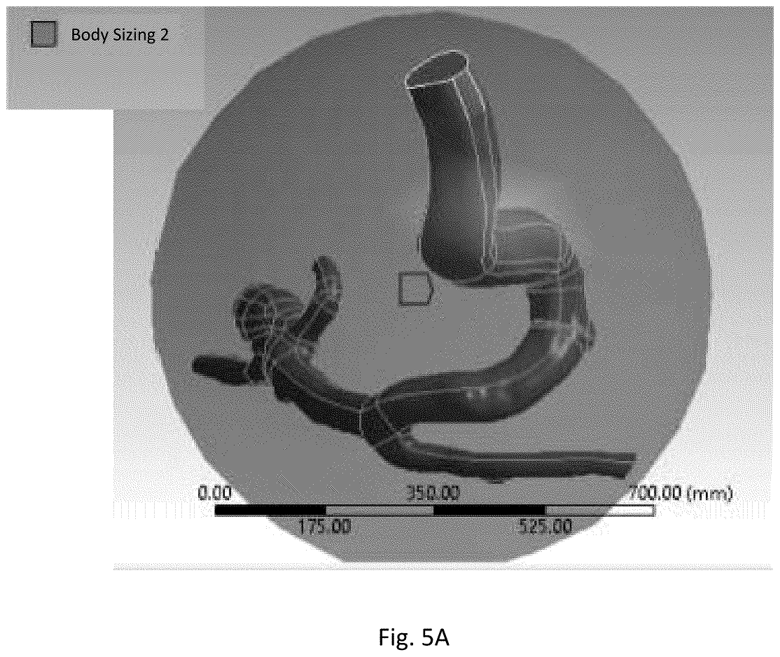

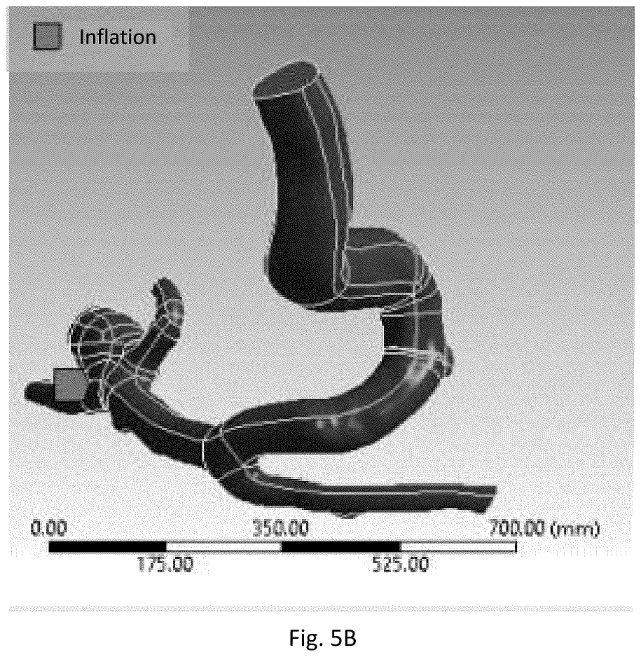

Mesh: In some embodiments, meshing is performed using the following two techniques: Body Sizing Mesh, and Inflation Meshing. FIG. 5A depicts the implementation of a body sizing mesh around a 3D model representing any of a region of interest (ROI), site of interest, a blood vessel, a cerebral aneurysm, and/or an intracranial aneurysm (IA) according to aspects of the present invention. FIG. 5B depicts the implementation of an inflation mesh within a 3D model representing any of a ROI, site of interest, a blood vessel, a cerebral aneurysm, and/or an intracranial aneurysm (IA) according to aspects of the present invention.

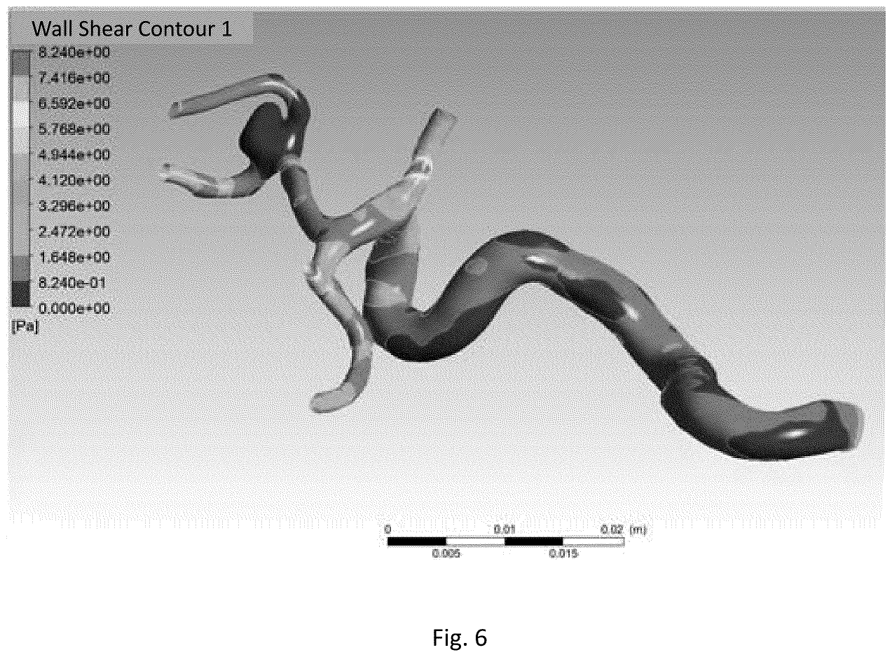

Fluent Solution Tool: In some embodiments, a fluent solution tool generates a simulation over specified cardiac cycles for the given boundary conditions. In some embodiments, the solution comprises various hemodynamic parameters of the blood flow such as Pressure, Velocity profile, and Wall Shear Stress (WSS). FIG. 6 is a plot showing the results for Wall Shear Stress Distribution across a 3D model according to aspects of the present invention.

Risk Assessment: In some embodiments, a risk assessment is performed using the data generated in ANSYS™. The data is exported as per the X, Y, and Z components of WSS which are further processed in MATLAB to generate the WSS indices such as Time Averaged Wall Shear Stress (TAWSS), Oscillatory Shear Index (OSI), and Relative Residence Time (RRT). These components are used to determine the growth, rupture risk, and progression of the aneurysm. The data is analyzed for statistical significance to verify its correlation with real-time assessment of the cases.

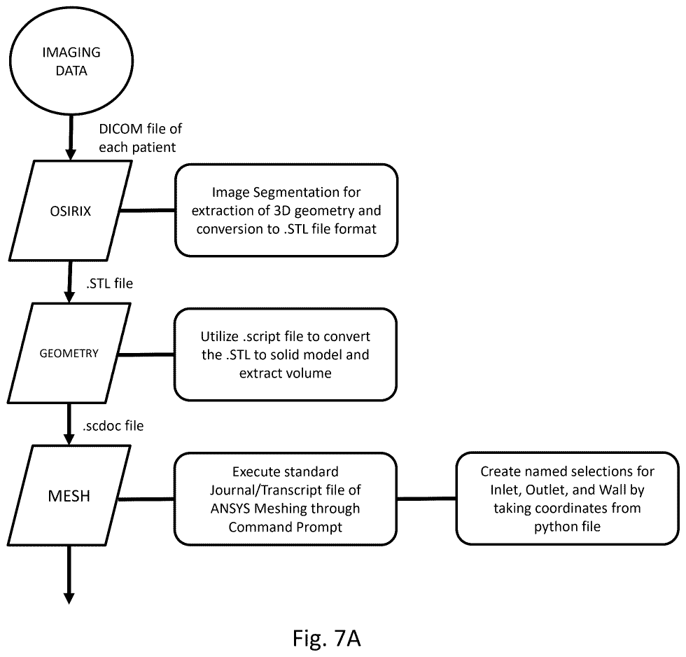

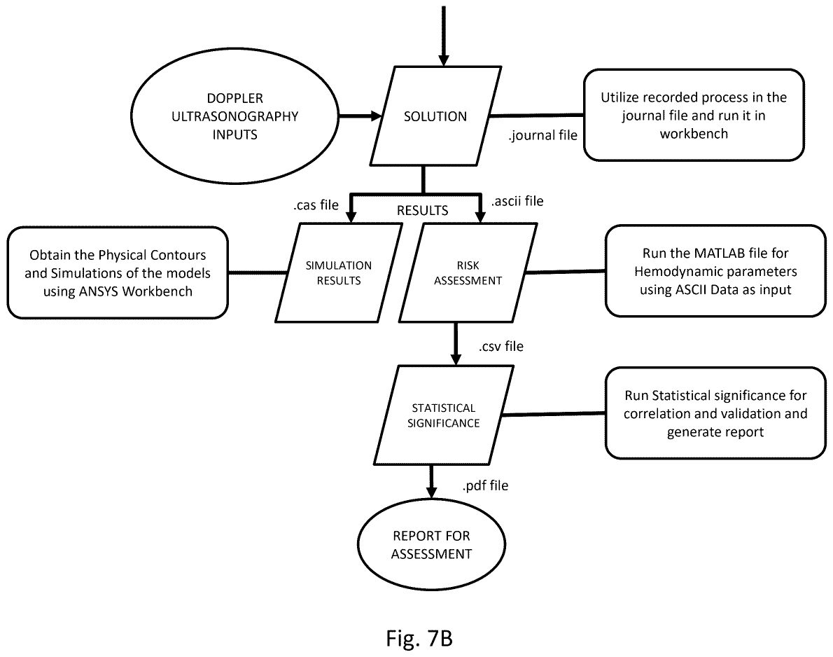

Software Integration: An exemplary software integration of the disclosed system and method is shown in FIGS. 7A & 7B. The light blue colored boxes represent the processes executed in the ANSYS™ platform. The Green-colored objects are the inputs and outputs of the system. The developed algorithm is intended to function such that minimum human intervention is required. Additionally, it has capabilities for the implementation of Artificial Intelligence and Machine Learning for adaptation as a smart assessment tool in clinical practice. The system is designed using known functions to reassure its validity. FIGS. 7A & 7B is a flowchart of an exemplary software integration for a method for assessing cerebral aneurysms according to aspects of the present invention.

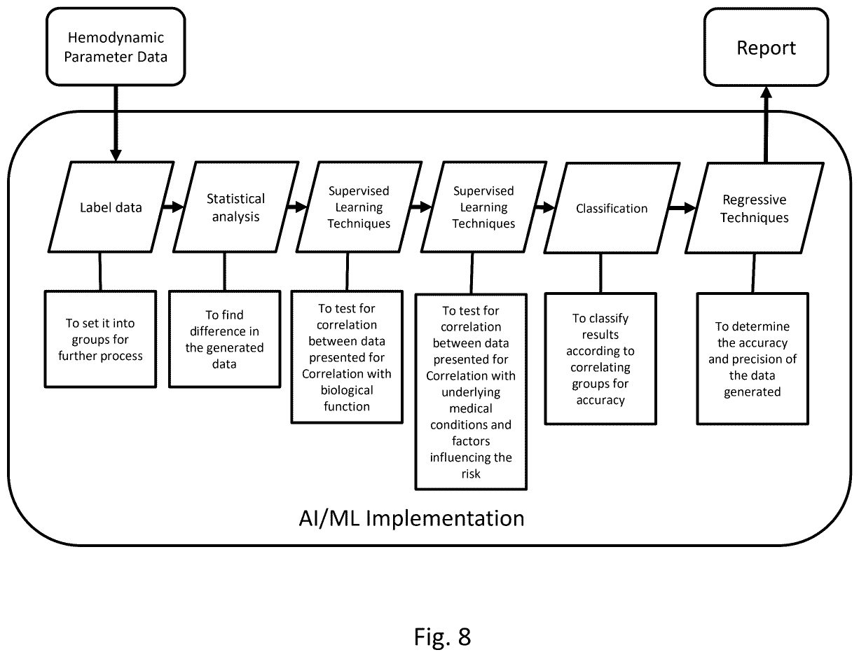

In order to assess reports accurately, Supervised AI/ML techniques were used that involved various forms of Correlation, Classification and Regression analysis. These techniques helped to determine the hemodynamic parameter data that is generated from the solution and provide insight into the underlying biological conditions of the vessels and walls, as well as the risk of rupture. Additionally, this data was used to evaluate the risk parameters against underlying factors such as Hypertension, smoking, age, medical history, and genetic predisposition that affect the condition for aneurysm. This approach helped to create more precise risk assessment reports and gain a better understanding of the affliction. FIG. 8 is a flowchart of an exemplary method for assessing cerebral aneurysms comprising Artificial Intelligence (AI)/Machine Learning (ML) according to aspects of the present invention.

The software disclosed serves as an assistive tool for clinical purposes, with potential applications that include:

Similar afflictions: The software can be used to study and monitor various afflictions such as stroke (Cardiovascular and Cerebral), subcranial hemorrhage, aneurysms in different areas such as abdominal, plaque deposition in arteries, as well as the study of blood vessels and arteries with the same hemodynamic parameters.

Stent implementation: The study can be further improved by assessing the implementation and effect of stents and their placement. This is achieved by introducing new steps that incorporate the geometry of stents into the design and generate a solution report on their impact on flow dynamics and effectiveness.

Clinical research: The software can also be used in clinical research for vascular implementation, particularly for conditions that can be assessed using similar hemodynamic parameters.

Vessel wall assessment: Additionally, the software can assess vessel walls, elasticity parameters, and study flow dynamics in vessel walls in general.

Overall the results of the example proved the validity of the disclosed methods, and the process was assured by comparing numerous different studies. The hemodynamic parameters data obtained from the models were consistently used for the determination of the risk of rupture, progression, growth, and other factors of the aneurysm in different studies. By utilizing these parameters, it became feasible to gain a thorough comprehension of the aneurysm.

### Example 2: Computational Analysis for Effects on Hemodynamic Parameters Based on the Location of Cerebral Aneurysms

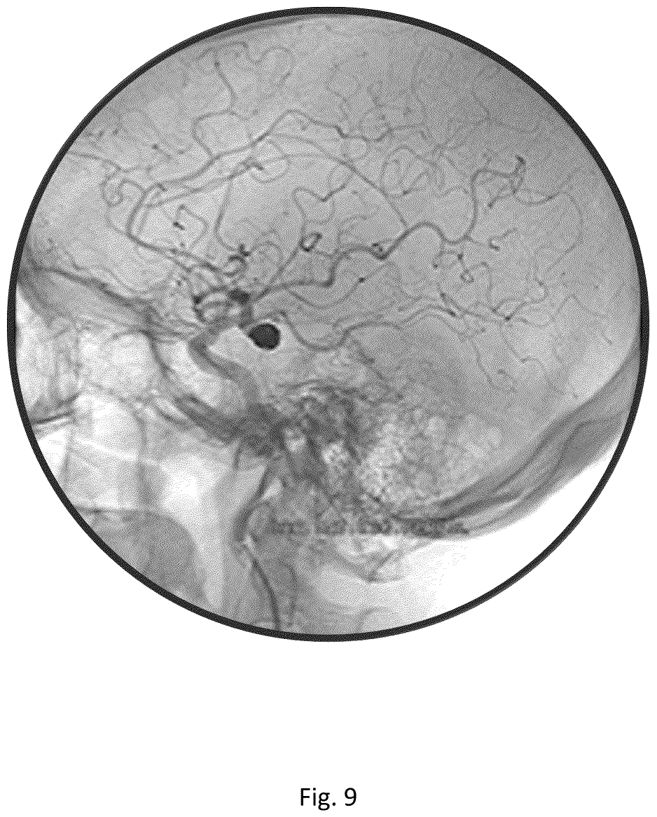

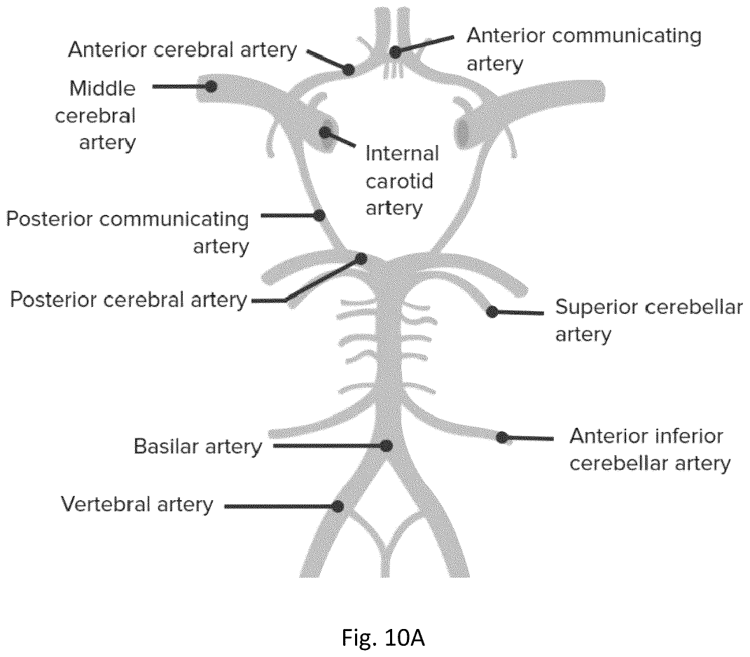

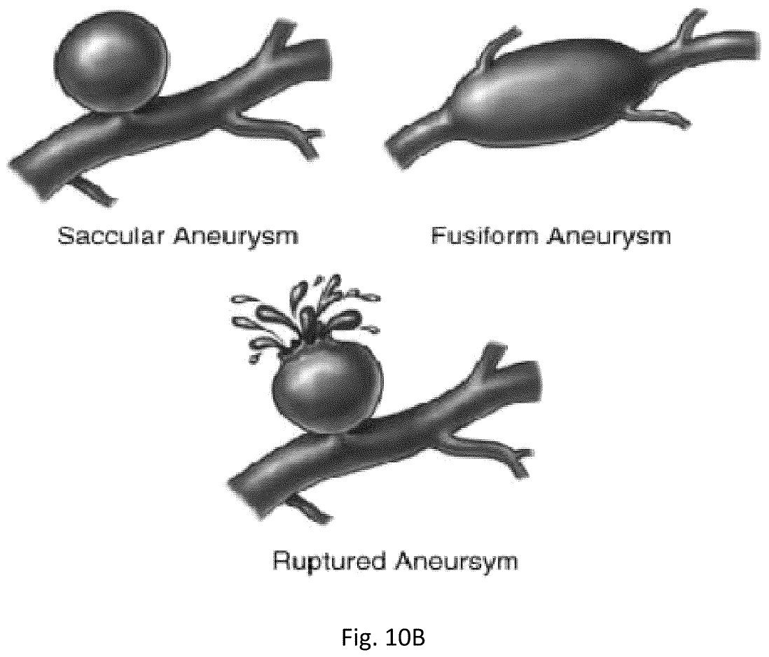

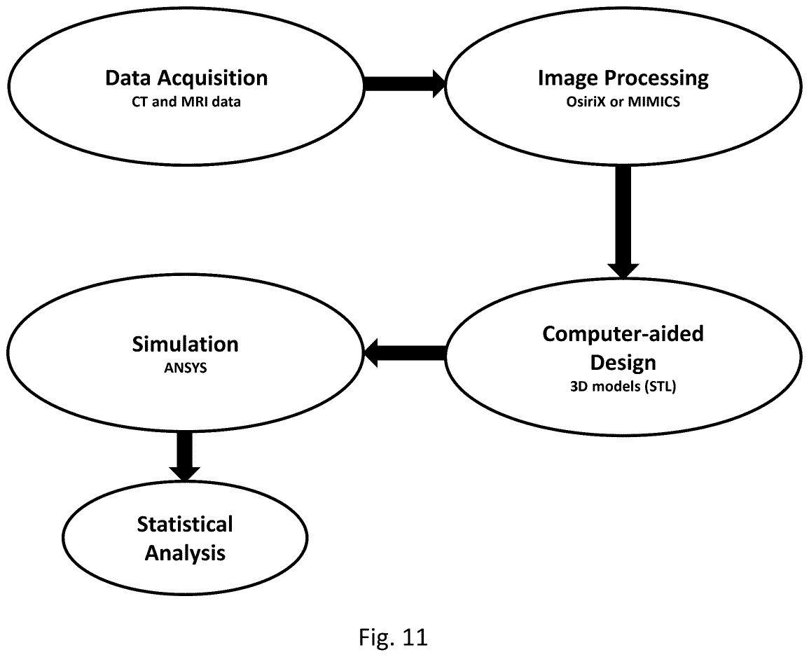

An aneurysm is a bulging in the artery's wall produced by the weakening of the muscles within the vascular wall. Since arteries transport blood under high pressure, they are prone to aneurysm development. Cerebral aneurysms can affect about 1.5 to 5 percent of the general population. The ratio of women:men=2:1 (>age 50). Factors contributing to the formation of Cerebral aneurysms are hypertension (high blood pressure), cigarette smoking, congenital (genetic) predisposition, injury or trauma to blood vessels, complications from some types of blood infections. FIG. 9 is an image depicting medical imaging of a cerebral aneurysm. The important locations in the brain where aneurysms can occur (see FIG. 10A) are Anterior Communicating Artery (ACA), Middle Cerebral Artery (MCA), Internal Carotid Artery (ICA), and Basilar Artery (BA). Exemplary types of cerebral aneurysms (see FIG. 10B) are Saccular (berry): Most common form present, occurs due to thin or absent Tunica media, commonly seen in the anterior circulation, Fusiform: Nonsaccular dilation of the whole circumference of the involved artery, mostly occurs in the basilar artery, and Mycotic: This a rare form that occurs secondary to infection of the vessel wall. Relevant Hemodynamic Parameters include Flow Velocity, Pressure Distribution, Wall Shear Stress (WSS), WSS-related indices: Time Average Wall Shear Stress (TAWSS), Oscillatory Shear Index (OSI) distribution, and Relative Residence Time. Relevant rupture Statuses include: Unruptured aneurysm, and Ruptured aneurysm. Other relevant factors considered for the study: Age, Presence of Multiple Aneurysms, Medical History. FIG. 11 is a flowchart depicting an exemplary method for assessing aneurysms. Table 1 below shows a model dataset.

TABLE 1
Model dataset for each location and medical history for a subject

| Model | | | | Aneurysm | Aneurysm | Rupture | Multiple | Medical |
|---|---|---|---|---|---|---|---|---|
| No. | Modality | Age | Sex | Type | Location | Status | Aneurysms | History |
| 1 | 3D-RA | 36 | M | TER | ACA | R | FALSE | N/A |
| 2 | 3D-RA | 47 | M | LAT | ICA | R | FALSE | N/A |
| 3 | 3D-RA | 53 | M | TER | MCA | R | FALSE | N/A |

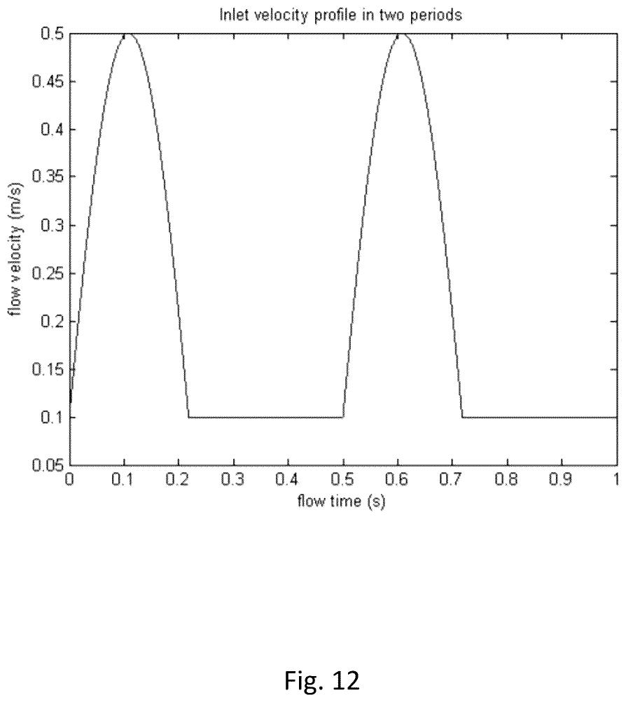

With regards to vessels in a subject and properties thereof described herein: Wall—the condition assumes that the velocity at the wall is zero due to the no-slip condition. Inlet—as is well known, mammalian blood flow is pulsatile and cyclic in nature. Thus, the velocity at the inlet is not set to be a constant, but instead, in this example, it is a time-varying periodic profile. The pulsatile profile within each period is a combination of two phases. During the systolic phase, the velocity at the inlet varies in a sinusoidal pattern. The sine wave during the systolic phase has a peak velocity of 0.5 m/s and a minimum velocity of 0.1 m/s. Assuming a heartbeat rate of 120 per minute, the duration of each period is 0.5 s. This model for pulsatile blood flow is disclosed by Sinnott et, al. [Sinnott, Matt & Cleary, Paul & Prakash, Mahesh. (2006). In Proc. Fifth International Conference on CFD in the Process Industries (pp. 1-6)]. A figure of the profile within two periods is given (see FIG. 12). Outlet—The systolic pressure of a healthy human is around 120 mm Hg, and the diastolic pressure of a healthy human is around 80 mm Hg. Thus, taking the average pressure of the two phases, 100 mm Hg (around 13,332 Pa) was used as the static pressure at the outlets. FIG. 12 shows a plot for the inlet velocity profile in two periods. The related velocity equation is below:

$${v}_{inlet} ( t ) = \{ \begin{matrix}0.5 sin [ 4 π ( t + 0.0160236 ) ] : 0.5 n < x \leq 0.5 n + 0.218 \\ 0.1 : 0.5 n + 0.218 < x \leq 0.5 ( n + 1 )\end{matrix} \qquad Equation 2$$

$$( n = 0 , 1 , 2 , .. )$$

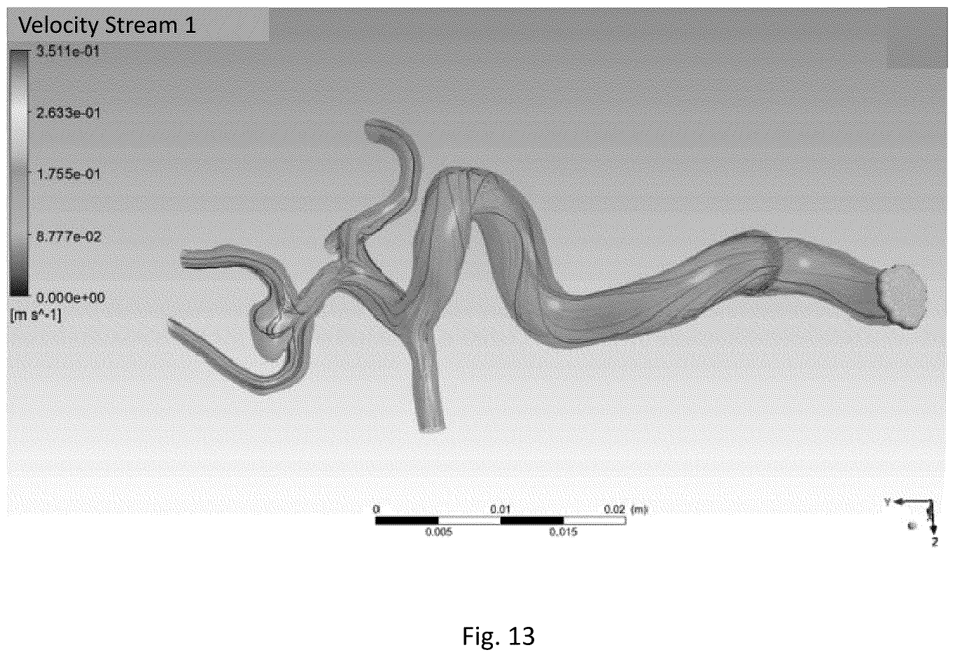

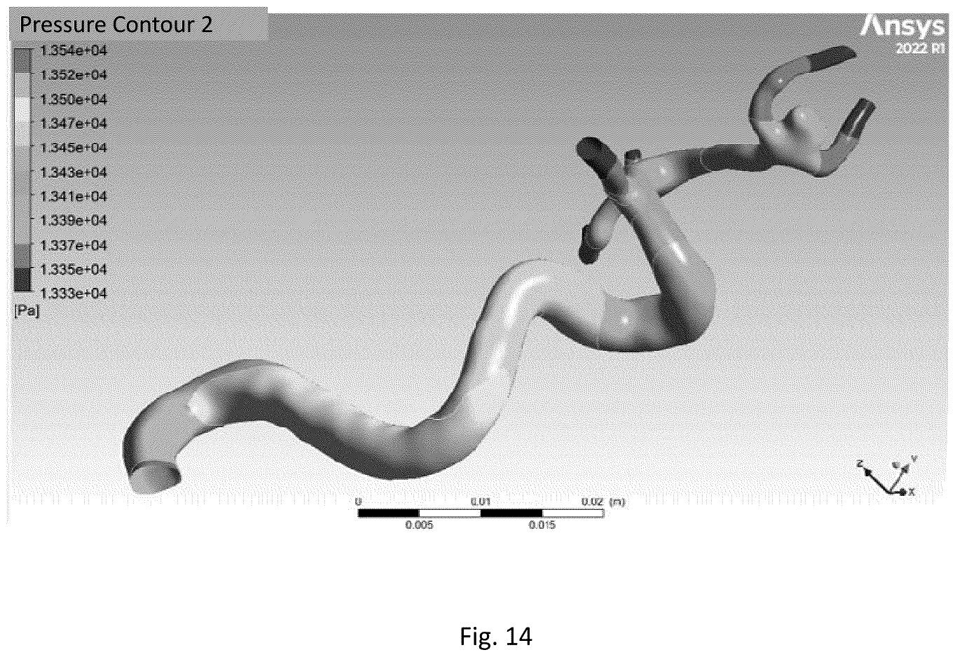

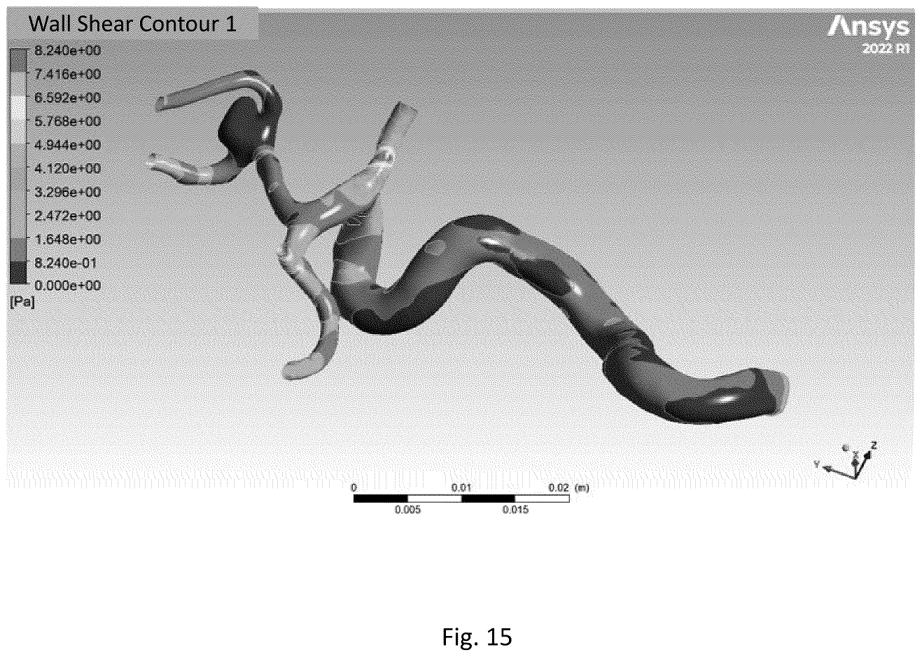

FIG. 13 is a plot showing a velocity profile for a 3D model of an ROI according to aspects of the present invention. FIG. 14 is a plot showing a pressure distribution for a 3D model of an ROI according to aspects of the present invention. FIG. 15 is a plot showing a wall shear stress distribution for a 3D model of an ROI according to aspects of the present invention.

Statistical Analysis: Four models pertaining to each location were analyzed. Using mean and standard deviation of the hemodynamic parameter values obtained from the CFD, a Jarque-Bera test for departure from a normal distribution analysis was performed to determine if a parameter was normally distributed. A 2-tailed independent Student t-test (for normally distributed data) was then performed for each parameter to assess the statistical significance of the observed difference between the mean values of the ruptured group. Probability values from the 2 tests were calculated and statistical significance would be assumed for P<0.01. The parameters that were significant (P<0.01) were then further analyzed using multivariate logistic regression (backward elimination) to identify those that retained significance when accounting for all relevant variables. Logistic regression were then performed on the significant variables (in the morphological category alone, hemodynamic category alone, and combined categories) to find final parsimonious models that allowed calculation of the quantitative risk of aneurysm rupture. This analysis was performed separately on the models pertaining to each location, which gave insights into the variations of the parameters based on the location.

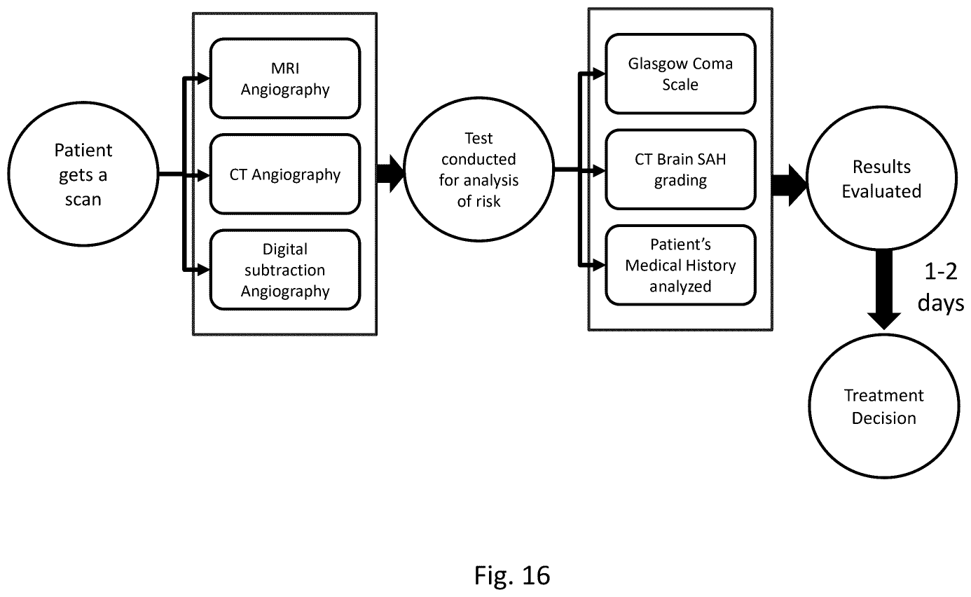

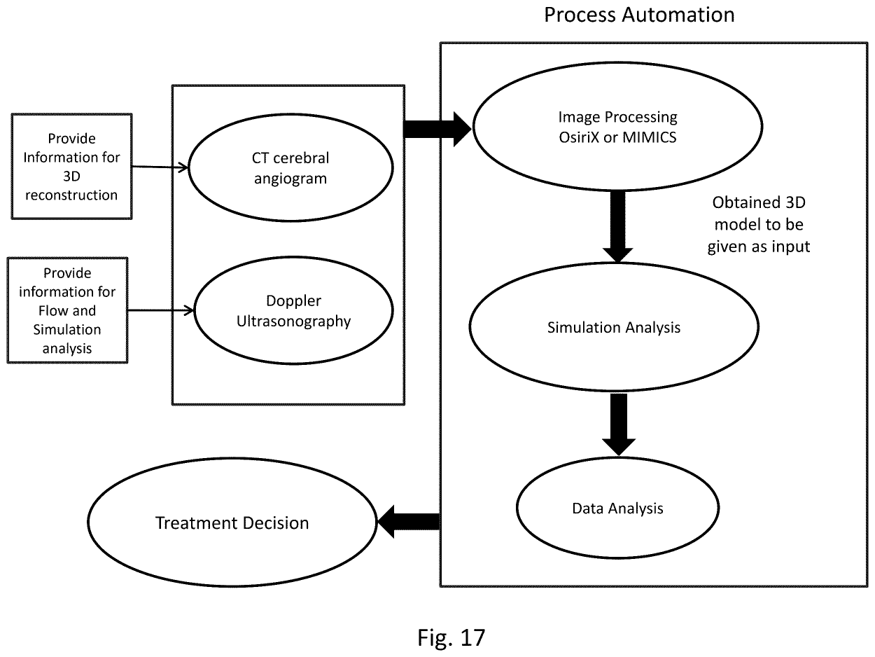

FIG. 16 is a flowchart depicting a diagnosis method for treatment prescription. This methodology is typically conducted over a period of days, involving various departments, and post analysis, a decision is made for treatment. FIG. 17 is a flowchart depicting an exemplary method for diagnosis of aneurysms in real-time according to aspects of the present invention. In some embodiments, the disclosed method may include a risk assessment of rupture probability, in-depth analysis of flow dynamics, shear force analysis and physical contours. The benefits of the disclosed methodology are that it provides faster diagnosis, assesses the risk of rupture, provides insights into physical modalities such as flow velocity, pressure analysis, and shear stress analysis, provides additional information to Physicians for making informed decisions, reduces workload and time by integrating processes into one system.

### Example 3: Automated System and Method for Intracranial Aneurysm (IA) Assessment and Treatment

In the field of neurosurgery, advanced imaging technologies are crucial in diagnosing and assessing Intracranial Aneurysm (IA). Patients undergoing treatment for cerebral aneurysms must undergo frequent scans, which can raise concerns about radiation exposure. One critical barrier in the current diagnosis methods is the limitations in the data provided, as up to 25% of patients seeking medical attention initially experience misdiagnosis or delays in diagnosis. Furthermore, an unmet need exists for a non-invasive technique that accurately determines blood flow characteristics. The absence of such methods not only drives up treatment costs but also elevates anxiety among patients. Hence, developing an affordable and non-invasive solution that offers insights into blood flow dynamics and predictive analysis of IAs holds immense potential to enhance the assessment process. The current methodology of using CT angiography (CTA) for detecting IA exhibits certain shortcomings, as indicated by the following results: The overall sensitivity and specificity of CTA per IA were 83% and 93%, respectively. This implies that CTA failed to detect 49 out of 284 IAs, resulting in a considerable false-negative rate. Among these missed IAs, 80% were small-sized (≤3 mm), making detecting such small lesions particularly challenging using CTA. Furthermore, CTA also struggled to detect IAs in the size range of 4-6 mm (18%) and 7-10 mm (2%). While the sensitivity and specificity of CTA per patient were higher at 95% and 97%, respectively, it was still found to miss primary IAs in 11 out of 211 patients.

These limitations, particularly for smaller aneurysms, raise concerns about the reliability of CTA as a standalone diagnostic tool for IA and underscore the need for complementary approaches to improve detection accuracy [Colen et al., American Journal of Roentgenology, 189(4), 898-903.]. Blood flow dynamics have several parameters to help understand how an IA behaves over time. Wall Shear Stress (WSS) is an essential parameter that describes the frictional force of viscous blood, which can affect endothelial cell function, gene expression, and cell shape and structure. There is growing evidence that WSS plays a crucial role in determining the progression of an aneurysmal disease [Satoh et al., AJNR Am J Neuroradiol. 2003; 24(7): 1436-1445; Wang et al., Neural Regen Res. 2013 Apr. 15; 8(11): 1007-15.]. The indices related to WSS, including Time Averaged Wall Shear Stress (TAWSS), Oscillatory Shear Index (OSI), and Relative Residence Time (RRT), are useful in assessing the Risk of Rupture (RoR), understanding its progression and the growth period of the IA. Previous studies in this area lack vital details, such as the patient's medical history, carotid artery blood pressure, and blood flow velocity, necessary for accurate simulation results. Therefore, by including the essential details required to generate an accurate patient simulation, the disclosed method described in this example provides clinicians with more precise results [Jiang et al., Annals of Vascular Surgery-Brief Reports and Innovations, 1(2), 100022; Brindise et al., Journal of the Royal Society Interface, 16(158), 20190465; Janiga et al., American Journal of Neuroradiology, 36(3), 530-536; Zhang et al., European Neurology, 66(6), 359-367; Meng et al., American Journal of Neuroradiology, 35(7), 1254-1262; Xiang et al., Stroke, 42(1), 144-152; Cebral et al., American Journal of Neuroradiology, 26(10), pp. 2550-2559; Paritala et al., Scientific Reports, 13(1), https://doi.org/10.1038/s41598-022-27354-w; Li et al., American Journal of Neuroradiology. https://doi.org/10.3174/ajnr.a6256]. This method solves many concerns by creating precise simulations for each patient scenario, removing doubts about the treatment plan. It also simplifies the current decision-making process and improves clinical practice. This allows physicians to better grasp the factors influencing each aneurysm case and make the right treatment decisions. Furthermore, the assessment reports generated are useful for doctors to streamline their workflow and save time [Qian et al., American Journal of Neuroradiology, 32(10), 1948-1955.].

Disclosed herein is an innovative approach in the field of Intracranial Aneurysm (IA) treatment with the development of an integrated and fully automated platform that has not existed before. This technology addresses the shortcomings of existing academic practices, significantly cutting down computational time and providing unparalleled customization for every patient. By incorporating crucial components necessary for in vivo simulations that were previously absent, the disclosed system and method is superior to current techniques in accuracy and efficiency. One of the innovative features of the disclosed system and method lies in the integration of Doppler ultrasonography, which furnishes precise velocity profiles for each patient, thereby enabling exceptionally accurate simulations [Purkayastha et al., Seminars in Neurology, 32(04), 411-420; Tremmel et al., Neurosurgery, 64(4), 622.]. The disclosed system and method harness the power of cutting-edge Artificial Intelligence (AI)/Machine Learning (ML) algorithms, which perform comprehensive risk assessments by meticulously analyzing the patient's complete medical history, alongside various physical and morphological factors associated with the IA. This advanced level of personalized risk evaluation allows for the formulation of tailored treatment plans, optimizing patient care [Shi et al., American Journal of Neuroradiology, 42(4), 648-654; Shi et al., American Journal of Neuroradiology, 41(3), 373-379; Ou et al., Frontiers in Neurology, 11. https://doi.org/10.3389/fneur.2020.570181; Yang et al., Journal of NeuroInterventional Surgery, 15(2), 200-204; Shi et al., American Journal of Neuroradiology, 41(3), 373-379.]. Ultimately, this technology culminates in generating a conclusive report that unequivocally validates the physician's treatment approach, instilling unparalleled confidence in clinical decision-making. This transformative and all-encompassing approach revolutionizes IA management, paving the way for improved patient outcomes and ultimately transforming the landscape of healthcare practices for years to come. Using CFD and 3D reconstructions to diagnose IAs significantly improves the accuracy of identifying the IA, potential risks, understanding blood flow patterns, and predicting potential RoR. This additional information allows medical professionals to make more informed decisions regarding the appropriate treatment approach. Regarding treatment, integrating CFD and 3D reconstructions in pre-operative planning leads to enhanced precision and personalized treatment options. By simulating the effects of different treatment strategies, medical teams may optimize the choice of procedures, such as surgical clipping, endovascular coiling, or flow diversion, tailored to each patient's specific case.

A pressing issue in the medical field is the need for a non-invasive risk assessment method for IA. Currently, available analysis methods rely heavily on invasive procedures such as CTA, Angiographies, and biopsies, which are painful and may cause adverse reactions in some patients. Furthermore, there needs to be more integrated information available to clinical practitioners regarding the assessment of IAs. In response to this need, the disclosed system and method provides a solution to provide clinical practitioners with a non-invasive approach to risk assessment for IAs. This CFD-based solution revolutionizes the field by offering a more patient-friendly assessment method, reducing the risk of adverse reactions, and providing an integrated platform for practitioners to access all the necessary information regarding IAs [Singla et al., Mathematical and Computational Applications, 28(4), 90; Murayama et al., Neurosurgical Focus, 47(1), E12]. Improving the efficiency of the workflow and minimizing the workload for doctors, allowing them to dedicate more time to their patients.

The disclosed system and method for assessing and treating intracranial aneurysms provides precise and conclusive results through non-invasive means by integrating and automating 3D reconstruction and CFD, surpassing the need for invasive procedures. Boundary conditions: Blood is a unique type of fluid, exhibiting a non-Newtonian fluid property. It has a pulsatile flow, miming the heart's pumping action. To streamline the modeling of non-Newtonian fluids, the governing equations used are continuity and Navier-Stokes, which are simplified further to fit into the Carreau model. Each model requires custom inputs for the flow velocity and pressure parameters obtained from the Doppler Ultrasonography for the boundary conditions. The wall conditions are given accordingly for all the models.

Process involved: In some embodiments, the CFD procedure was carried out in three stages using the ANSYS™ platform. Specific details regarding the methods and steps used in the disclosed processes can be found in: [Silva et al., Surgical Neurology International, vol. 13, Scientific Scholar, April 2022, p. 182. Crossref; Sankar et al., Abstract and Applied Analysis, vol. 2012, Hindawi Limited, 2012, pp. 1-34. Crossref; Lopes et al., International Journal of Mechanical Sciences, vol. 160, Elsevier BV, September 2019, pp. 209-18. Crossref; Berg et al., Neurosurgical Focus, vol. 47, no. 1, Journal of Neurosurgery Publishing Group (JNSPG), July 2019, p. E15. Crossref; Lopes et al., European Journal of Mechanics-B/Fluids, vol. 83, Elsevier BV, September 2020, pp. 226-34; Crossref; Hasan et al., Hypertension, vol. 66, no. 2, Ovid Technologies (Wolters Kluwer Health), August 2015, pp. 324-31. Crossref; Karmonik et al., Acta Neurochirurgica, 151(5), 479-485.]

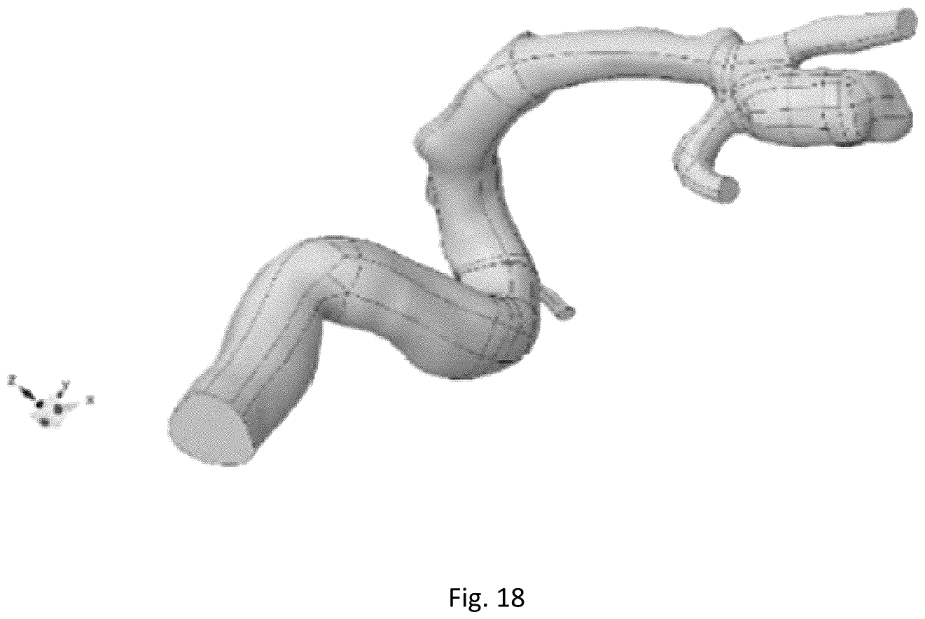

Space Claim: The process of extracting volume was accomplished by utilizing SpaceClaim, ANSYS™ software. The reconstructed 3-D file was then passed to SpaceClaim, which was presented in facets. By employing the 3-D modeling capabilities, the faceted body was transformed into a solid model. Once the solid model was achieved, the volume was then extracted accordingly. FIG. 18 depicts a solid 3D model for the site of an intracranial aneurysm (IA).

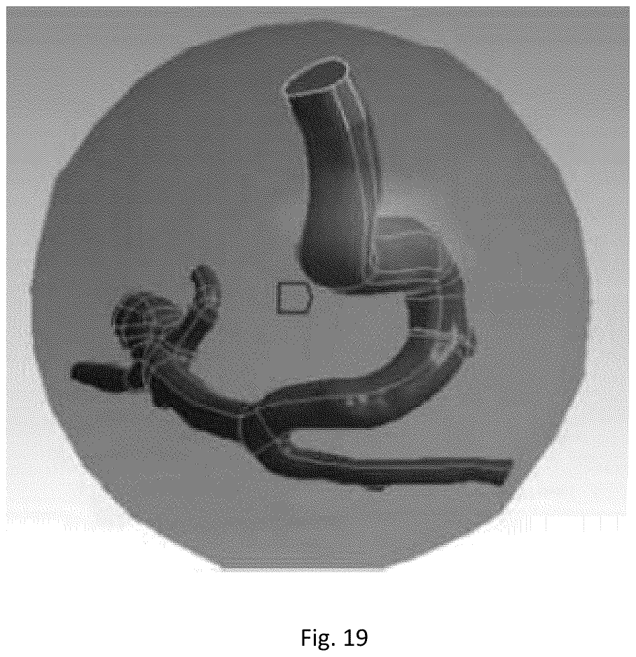

Mesh: Meshing was performed using the following techniques: Body Sizing Mesh and Inflation Meshing. Different element sizes were tested to determine the optimal mesh size. The mesh results were compared using 0.1 mm element size as the original case and 0.5 mm element size as refined. By comparing the values, it was observed that the difference between the original case and the refined case for velocity is only 0.02%. The pressure value was consistent in both cases. Thus, the finer mesh value of 0.1 mm has been chosen as the element size for better results, although a coarse mesh would be acceptable. FIG. 19 shows the implementation of a body sizing mesh around a solid model of an IA according to aspects of the present invention.

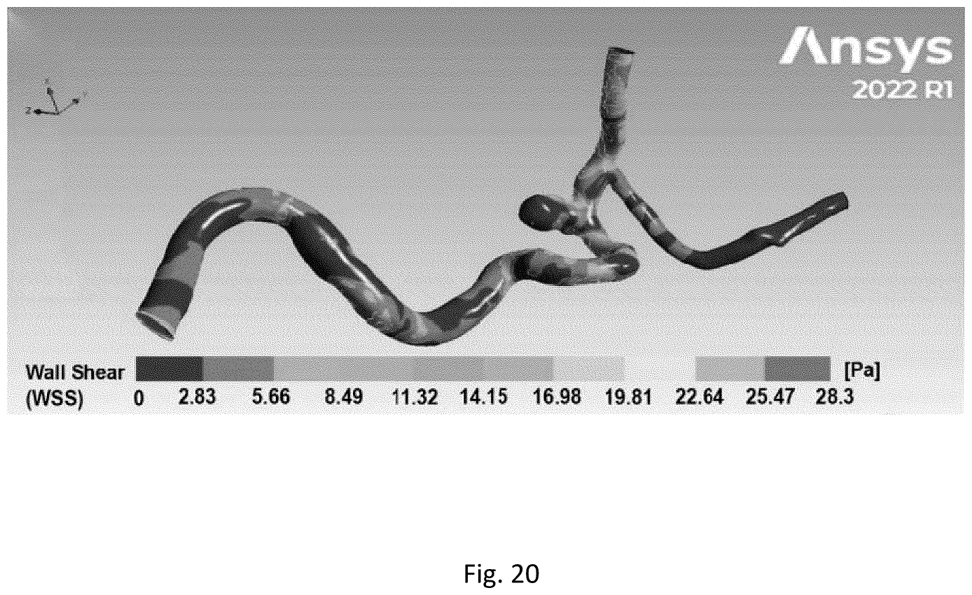

Fluent Solution Tool: The fluent solution tool generates a simulation over specified cardiac cycles for the given boundary conditions. The solution includes various hemodynamic parameters of the blood flow, such as Pressure, Velocity, and Wall Shear Stress (WSS) distributions at the point of interest. An example is represented in FIG. 20. FIG. 20 shows a plot for Wall Shear Stress (WSS) distribution across the walls of the ruptured IA according to aspects of the present invention.

The disclosed system and method automate and integrate the software involved, with the resources to generate customized solutions for each case and reduce computational time. In some embodiments, the disclosed method comprises image segmentation for the CT/MRI data and producing simulation results through solution analysis. In some embodiments, the system and method comprises an amalgamation of systems and software, and access to a platform or graphical user interface (GUI) is provided through a single framework, in some embodiments comprising additional licensed software. The steps associated with each process was established in three distinct stages, each with its own objectives and corresponding actions.
Stage 1: Automation of Image Segmentation process to get 3-D reconstructions from Medical Imaging data: Creating 3-D models from advanced imaging scans was crucial for the disclosed approach. A licensed platform was used for image segmentation to ensure the reconstructed blood vessels' accuracy. OsiriX™ is the preferred software for this task, allowing the conversion of DICOM images to 3-D geometry in (.STL) format. By utilizing OsiriX™'s Plugin-Development-Framework, the workflow was streamlined for greater efficiency. The disclosed method comprises importing the DICOM images into OsiriX™, setting a pixel thresholding value for optimal visibility of the blood vessels, and highlighting the region of interest in the image. Next, the volume generation process was executed, followed by smoothening using the available tools for creating the (.STL) model. This process generates highly accurate 3-D models of blood vessels. Using the Plugin-Development-Framework of OsiriX™, the image-segmentation process was automated through OsiriXAPI.framework.
Stage 2: Automation for Geometry and Mesh adjustment for reconstruction of cerebral arteries: For the CFD aspect of the disclosed system and method, ANSYS™ was utilized, a second licensed software. The SpaceClaim platform on ANSYS™ allows recording and scripting the steps involved in processing the 3-D faceted model into a solid body and extracting the Volume for meshing purposes. The extracted volume was then passed through the Meshing application on the Fluent workbench as a standard process by journal scripts for automating the workflow. The different faces of the blood vessels were then defined as the inlet, outlet, and wall of the artery. The files generated from the Meshing application were then processed to Solution Setup.
Stage 3: Automated solution generation for Navier-Stokes modeling the blood flow through arteries: The solution setup in ANSYS™ allows workflow automation through its plugin development. The boundary conditions discussed earlier are the foundation for the blood flow through the arteries. The blood flow is modeled using Navier-Stokes equations to imitate the properties of blood. The solution is designed to operate for any desired cardiac cycle. This step accurately generates the shear force values and the blood flow dynamics. The results of the solution generated were extracted in ASCII format for further analysis, along with the blood flow simulations through the reconstructed arteries. To accurately customize the blood flow for patient-specific cases, the disclosed solution provides a customized velocity waveform and blood pressure for each case. The generated results provided information about the fundamental hemodynamic parameters such as WSS, Flow velocity, and pressure distribution.

In some embodiments, the hemodynamic parameters generated from the tool will provide invaluable insights into the behavior of IAs and their RoR. Another result of the disclosed example was the development of an AI/ML model to evaluate the RoR and provide essential insights into the behavior of IAs over time, which is accessible to clinicians and physicians.

The disclosed system and method provides a comprehensive risk assessment framework for IAs through a robust AI/ML model with a systematic approach to data collection, feature extraction, model development, evaluation, and future enhancement. The disclosed methodology is outlined in six stages that collectively contribute to the proposed improved risk assessment framework.

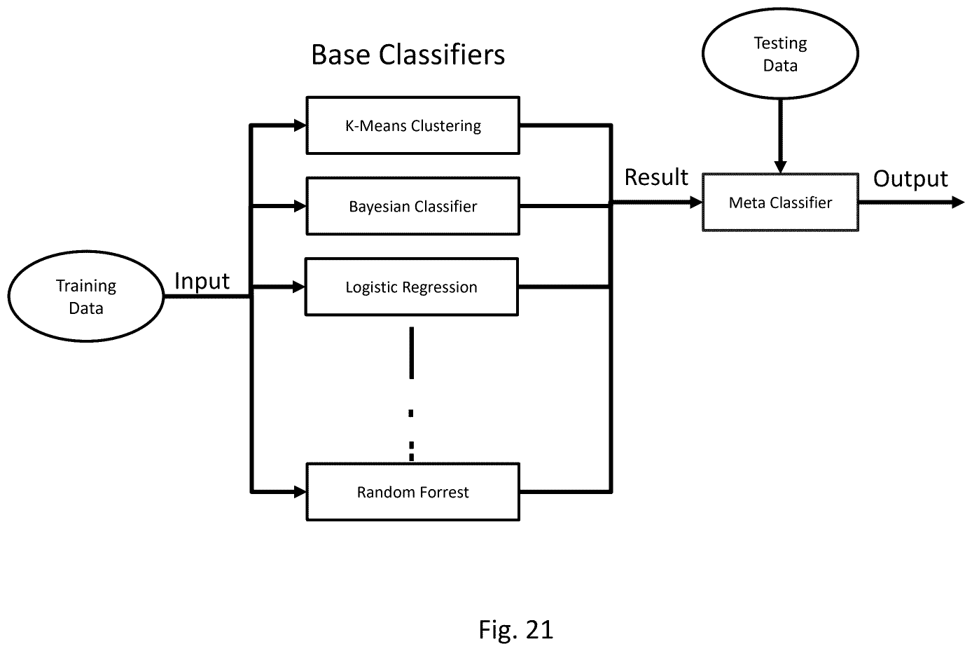

In some embodiments, the disclosed system and method identifies 20+key features influencing the RoR from the data set, outlines a scale for the risk determination thereof, and compares the results to the established standard such as PHASES Score. In the disclosed example, the system and method achieved a cross-validated Area Under the Curve of the Receiver Operating Characteristic Curve (AUC-ROC) score of 0.75 or more on an independent validation dataset, showcasing its ability to categorize risk degrees accurately and compute a model accuracy of ˜90%.
Stage 1: Data Acquisition and Integration: (1) Calculating Morphological features: Morphological and physical analysis of IA was done manually from the 3-D reconstructed geometry for the volume of the aneurysmal sac, size of IA, the diameter of the parent artery, etc., and derived features were further calculated in the feature engineering process. (2) Calculating Hemodynamic features: The ASCII data from the ANSYS™ solution provided WSS, Pressure, and Flow dynamics. The hemodynamic features OSI, RRT, and TAWSS are derivatives of WSS that are calculated from mathematical expressions. (3) Extracting Patient Health and History: Medical records contain valuable information, including doctor's notes and comments, which can provide insights into a patient's medical and family history, as well as symptoms related to IAs. Natural Language Processing (NLP) techniques, such as rule-based techniques, can extract keywords from these notes to summarize them and identify topics of interest that serve as the data points.
Stage 2: Feature Exploration and Selection: in order to train the disclosed machine-learning model effectively, gathering and analyzing data meticulously for each feature and its corresponding risk factors was imperative. The raw data was manipulated and transformed to suit the purpose of the model. A correlation matrix was created through data exploration to understand the dataset better. This process identified significant correlation between the Rupture status and the SizeRatio (P<0.001). The SizeRatio is calculated as the ratio of the IA size to the diameter of the parent artery. Furthermore, it was discovered that the location of the IA is a crucial factor (P<0.001). It is worth noting that patients with an IA size below the conventional risk threshold (<7 mm) are more likely to experience early rupture if the location is Anterior Community Artery (AcomA). These causal relationships served as a foundation to filter out deterministic features while paving the way for other features that can be derived from the existing ones. As the AI/ML model for aneurysm risk determination evolved, feature exploration encompassed a broader range of variables, enriching the predictive capabilities. Additional data dimensions for patient demographics, medical history, and hemodynamic parameters were integrated. This expansion facilitated discovering intricate relationships between risk factors and IAs rupture, enhancing the model's accuracy and clinical relevance. The model unveiled previously unseen correlations through meticulous data collection and integration, contributing to a more comprehensive risk assessment. This iterative approach ensured that the AI/ML model's insights continually deepened, reflecting the complex interplay of factors influencing IA behavior and risk, empowering healthcare professionals.
Stage 3: Feature Engineering and Model Input: Raw features in the dataset were refined to establish better causal relationships among key features. This proved relevant relationships among risk factors and provided key features to train the risk prediction model. For example, the SizeRatio, a pivotal metric in the analysis, was calculated as the ratio of aneurysm size to the diameter of the parent artery. When considering the morphological factors of an IA-like Sac Volume, it would be interesting to think that Sac volume would be higher for ruptured IA. Still, there could be no significant relationship established between Rupture status and the Sac Volume (P=0.51). It is clear from the analysis conducted that the sac's size or volume would not be a significant factor unless the size ratio is considered. When considering two aneurysms with almost the same maximum diameters (4.8 mm) but in different locations, the AcomA and Middle Cerebral Artery, their risk of rupture changes as the parent vessel diameter is smaller in the AcomA, thus increasing the RoR. The developed features were further tested to determine the statistical significance. The continuous process of improving the features of a model through feature engineering led to a better understanding of patterns and connections that were previously unnoticed, ultimately enhancing the development of the model's predictive capabilities.
Stage 4: Model Development and Ensemble: Using the mean and standard deviation of the hemodynamic parameter values obtained from the CFD, a Jarque-Bera test for departure from a normal distribution analysis were performed to determine if a parameter is normally distributed. This was followed by a 2-tailed independent Student t-test (for normally distributed data) for each parameter to assess the statistical significance of the observed difference between the mean values of the ruptured group. Probability values from the two tests were calculated, and statistical significance was assumed for P<0.01. The significant parameters (P<0.01) were then further analyzed using multivariate logistic regression (backward elimination) to identify those that retained significance when accounting for all relevant variables. Logistic regression was then performed on the significant variables (in the morphological category alone, hemodynamic category alone, and combined categories) to develop category-specific parsimonious models that provided insights into feature-specific influence on the RoR [Silva et al., World Neurosurgery, 131, e46-c51; Tanioka et al., Radiology: Artificial Intelligence, 2(1), e190077.]. The accuracy of the models classified falls along the same range, but the approach was more reliable when these models were ensembled to perform collectively toward the classification problem. Multiple classifiers were selected such as Logistic Regression, Decision Trees, K-means clustering, and Gaussian NB Classifier to get classified labels from the dataset. The test set performed individually for these base models was decent enough for the ensemble. A stack classifier could be considered as a Meta-classifier that takes the inputs from these base classifiers set for a final prediction task. Stack classifiers have been known to outperform state-of-the-art models and improve total predictive performance. FIG. 21 is a flowchart depicting an exemplary training model comprising a stack classifier according to aspects of the present invention. Several classification models were performed that have provided insights into the significance of the variables encountered. The average P-value of the variables, IA Location—0.018, Size Ratio—0.014, Sac Volume—0.05. These three variables proved to be significant in the morphological study of the IA. By individually considering Hemodynamic, Morphological, and Medical History features for the disclosed parsimonious models, profound risk assessment can be achieved. Incorporation of these features into the final model enhanced precision of risk analysis and RoR metric [Rayz et al., Annual Review of Biomedical Engineering, 22(1), 231-256; Amigo et al., Journal of Engineering in Medicine, 235(6), 655-662; Paliwal et al., Neurosurgical Focus, 45 (5), E7.].
Stage 5: Evaluation and Assessment: For model evaluation, several established metrics were utilized to measure its effectiveness. These metrics included AUC-ROC, precision-recall curve, G-score, and F1-score. Additionally, essential metrics such as accuracy, recall, specificity, and sensitivity were used. Recursive evaluation was employed to determine the significance of features in stratification. To test the model's overall accuracy, it was compared against the established standard metric for IA risk determination, the PHASES score. This score considers several factors associated with risk, such as morphological and patient data. The disclosed analysis was evaluated by comparing the model accuracy against standards such as PHASES (Population, Hypertension, Age, Size, earlier subarachnoid hemorrhage, and Site), UIATS (Unruptured Intracranial Aneurysm Treatment Score), ICUIA (International Study on Unruptured Intracranial Aneurysms), and UCAS (Unruptured Cerebral Aneurysm Study) to determine its effectiveness and reliability. Achieving interpretability in AI/ML models for IAs risk determination involves many strategies that enhance transparency and provide invaluable insights [ISUIA (International Study on Unruptured Intracranial Aneurysms), The Lancet 2003, doi: 10.1016/s0140-6736 (03) 13860-3; UCAS (Unruptured Cerebral Aneurysm Study in a Japanese Cohort); New England Journal of Medicine 2012; doi: 10.1056/NEJMoal113260; PHASES, The Lancet Neurology 2014, doi: 10.1016/S1474-4422 (13) 70263-1; unruptured intracranial aneurysm treatment score (UIATS), Neurology 2015, doi: 10.1212/WNL.0000000000001891]. Through feature importance analysis, the pivotal factors were uncovered that shape predictions and quantify the influence of each feature. With visual explanations in the form of heat maps, bar charts, and line plots, intuitive depictions of complex relationships were provided. Partial Dependence Plots (PDPs) demonstrated how changes in individual features impact overall risk, offering granular insights. Techniques like Local Interpretable Model-agnostic Explanations (LIME) facilitated individual instances and counterfactual explanations, generating interpretable surrogate models for personalized predictions. Contextual interpretation anchors predictions in medical domain knowledge, showcasing interactions between conditions, histories, and model outcomes. To mitigate potential model bias and imbalance during data training, measures were implemented such as the K-fold cross-validation method and hyperparameter tuning. These steps ensured that the model performance remained consistent across all test sets. Furthermore, conducting several hypothesis tests, like McNemar's test and the Wilcoxon signed-rank test accurately compared the performances of each test set.
Stage 6: Clinical Decision Support and Documentation Reporting: At this final stage, the clinicians thoroughly reviewed the risk assessment results and other clinical information. This allowed physicians to make well informed decisions about the diagnosis, treatment, or additional tests that may be necessary. Following the physician's approval, a comprehensive report was generated for thorough examination and analysis.

In some embodiments, the disclosed method may utilize machine learning methods such as Hyper Parameter Tuning to engineer the best combination of factors and create an accurate model that aligns with the actual data. In some embodiments, to deal with the dataset with fewer rows, dimensionality reduction techniques may be applied like Principal Component Analysis (PCA) and Linear Discriminant Analysis (LDA) to transform data into lower-dimensional space while preserving variance. If the classification methods are ineffective in raising the bar, regularization techniques may be utilized to maintain the context of the data while shrinking down less relevant data points. These techniques do not change classification results significantly, but speed up computation without adding too many features, and avoid overfitting.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for assessing cerebral aneurysms, comprising the steps of:
collecting imaging and blood flow data of an aneurysm and the attached arteries in a subject;
creating a 3D model of the aneurysm and the attached arteries from the imaging data, wherein creating the 3D model comprises representing the attached arteries as an arterial volume comprising at least one arterial inlet and at least one arterial outlet, applying a fluid governing equation to the arterial volume comprising the Navier Stokes equation, and applying arterial inlet and arterial outlet boundary conditions to the fluid governing equation based on the blood flow data of the attached arteries;
generating blood flow simulations with the 3D model and the blood flow data;
extracting one or more parameters from the simulations;
processing the one or more parameters to generate one or more stress indices; and
calculating a risk assessment score for the subject based on a statistical analysis of the one or more parameters and the one or more stress indices.

2. The method of claim 1, wherein the imaging data is collected from at least one of CT Cerebral Angiogram, MRI, MRA, 3D DSA or 4D DSA.

3. The method of claim 2, wherein the blood flow data comprises real-time flow velocity and pressure data from Doppler Ultrasonography.

4. The method of claim 3, wherein the one or more parameters comprise a hemodynamic parameter and/or a morphological parameter.

5. The method of claim 4, wherein the hemodynamic parameter is selected from the group consisting of: Pressure, Pressure Distribution, Flow Velocity, Velocity profile, Wall Shear Stress (WSS), mean maximum WSS (MWSS), mean parent vessel WSS (PTWSS), mean normalized WSS (NWSS), WSS gradient (WSSG), Transverse WSS (TWSS), Aneurysm formation indicator (AFI), Oscillation velocity index (OVI), Gradient oscillatory number (GON), Relative residence time (RRT), or mean oscillatory shear index (OSI).

6. The method of claim 5, wherein the morphological parameter is selected from the group consisting of: aneurysm size, aspect ratio (AR), size ratio (SR), ellipticity index (EI), undulation index (UI), nonsphericity index (NSI), shape of aneurysm, bottleneck factor (BNF), vessel angle, parent artery diameter, aneurysm neck width, aneurysm height, or aneurysm width.

7. The method of claim 6, wherein the stress indices comprise any of Time Average Wall Shear Stress (TAWSS), Oscillatory Shear Index (OSI), and Relative Residence Time (RRT).

8. The method of claim 7, wherein the step of calculating the risk score comprises a statistical analysis to correlate at least one of the one or more parameters and stress indices with one or more medical factors of the subject.

9. The method of claim 8, wherein the one or more medical factors comprise any of hypertension, sex, smoking, age, medical history, aneurysm type, aneurysm location, rupture status, multiple aneurysms, genetic predisposition affecting the aneurysm condition, injury or trauma to blood vessels, complications from some types of blood infections, blood lipid levels, glucose levels, and indication for diabetes.

10. The method of claim 9, further comprising the step of treating the subject with one or more treatments based on the outcome of the risk assessment score.

11. The method of claim 10, wherein the treatment comprises implantation of a stent.

12. The method of claim 11, further comprising the step of assessing the implementation and effectiveness of stent by repeating any previous steps to calculate a treatment assessment score.

13. (canceled)

14. The method of claim 13, wherein the step of generating the blood flow simulation comprises providing one or more data inputs from flow velocity and pressure values of the blood flow data.

15. The method of claim 1, wherein the statistical analysis is performed in a neural network trained with imaging and blood flow data associated with vessel occlusion.

16. A system for assessing cerebral aneurysms in a subject, comprising a non-transitory computer-readable medium with instructions stored thereon, that when executed by a processor perform the steps of:
collecting imaging and blood flow data of an aneurysm and the attached arteries in a subject;
creating a 3D model of the aneurysm and the attached arteries from the imaging data, wherein creating the 3D model comprises representing the attached arteries as an arterial volume comprising at least one arterial inlet and at least one arterial outlet, applying a fluid governing equation to the arterial volume comprising the Navier Stokes equation, and applying arterial inlet and arterial outlet boundary conditions to the fluid governing equation based on the blood flow data of the attached arteries;
generating blood flow simulations with the 3D model and the blood flow data;
extracting one or more parameters from the simulations;
processing the one or more parameters to generate one or more stress indices;
calculating a risk assessment score for the subject based on a statistical analysis of the one or more parameters and the one or more stress indices.

17. The system of claim 16, wherein the imaging data may be collected from any of CT Cerebral Angiogram, MRI, MRA, 3D DSA, or 4D DSA.

18. The system of claim 17, wherein the blood flow data comprises real-time flow velocity and pressure data from Doppler Ultrasonography.

19. The system of claim 18, wherein the one or more parameters comprise a hemodynamic parameter and/or a morphological parameter.

20. The system of claim 19, wherein the hemodynamic parameter is selected from the group consisting of: Pressure, Pressure Distribution, Flow Velocity, Velocity profile, Wall Shear Stress (WSS), mean maximum WSS (MWSS), mean parent vessel WSS (PTWSS), mean normalized WSS (NWSS), WSS gradient (WSSG), Transverse WSS (TWSS), Aneurysm formation indicator (AFI), Oscillation velocity index (OVI), Gradient oscillatory number (GON), Relative residence time (RRT), or mean oscillatory shear index (OSI).

21. The system of claim 20, wherein the morphological parameter is selected from the group consisting of: aneurysm size, aspect ratio (AR), size ratio (SR), ellipticity index (EI), undulation index (UI), nonsphericity index (NSI), shape of aneurysm, bottleneck factor (BNF), vessel angle, parent artery diameter, aneurysm neck width, aneurysm height, or aneurysm width.

22. The system of claim 20, wherein the stress indices comprise any of Time Average Wall Shear Stress (TAWSS), Oscillatory Shear Index (OSI), and Relative Residence Time (RRT).

23. The system of claim 16, further comprising a neural network trained with imaging and blood flow data associated with vessel occlusion, wherein the statistical analysis is performed with the neural network.
